(19) United States
(12) Patent Application Publication
(10) Pub. No.: US 2024/0105298 A1
(43) Pub. Date: Mar. 28, 2024

(54) MACHINE LEARNING MODELS FOR DESIGNATION OF SUBJECTS FOR TREATMENT AND/OR EVALUATION

(71) Applicant: Mor Research Applications Ltd., Ramat-Gan (IL)

(72) Inventors: Doron NETZER, Ramat Gan (IL); Ran BALICER, Ramat Gan (IL); Noa DAGAN, Ramat Gan (IL); Eldad KEPTEN, Ramat Gan (IL); Nava LEIBOVICH, Ramat Gan (IL); Yifat MIRON, Ramat Gan (IL); Shlomit BLOOMENTHAL, Ramat Gan (IL); Jacob Gershon WAXMAN, Ramat Gan (IL); Ronit SAFAR, Ramat Gan (IL); Moria MAHANAIMY, Ramat Gan (IL); Ilana ROITMAN KALISHOV, Ramat Gan (IL)

(73) Assignee: Mor Research Applications Ltd., Ramat-Gan (IL)

(21) Appl. No.: 18/283,236

(22) PCT Filed: Mar. 20, 2022
(86) PCT No.: PCT/IB2022/052517

§ 371 Date: Sep. 21, 2023

(30) Foreign Application Priority Data
Mar. 22, 2021 (IL) 281746

(51) Int. Cl. G16H 20/00; G16H 10/60; G16H 50/20; G16H 50/30

(52) U.S. Cl. CPC G16H 20/00; G16H 10/60; G16H 50/20; G16H 50/30

(57) ABSTRACT

A method comprises: for each specific medical intervention and/or respective clinical outcome: accessing a respective specific priority list of a respective sub-set of subjects scheduled in a prioritized sequence for treatment and/or evaluation, creating a respective specific training dataset that includes data extracted from EMRs of the respective sub-set of subjects labelled with the specific priority list, and training a respective specific machine learning model on the respective specific training dataset for generating an outcome of a respective specific priority list of a sub-set of subjects for prioritized evaluation and/or treatment, in response to an input of data extracted from EMR of the sub-set of subjects, accessing a combined prioritization component for generating an outcome of a combined priority list of subjects for prioritized evaluation and/or treatment in response to an input of outcomes of the specific machine learning models, and providing the specific models and the combined prioritization component.

### RELATED APPLICATION

This application claims the benefit of priority of Israeli Patent Application No. 281746 filed on Mar. 22, 2021, the contents of which are incorporated herein by reference in their entirety.

### BACKGROUND

The present invention, in some embodiments thereof, relates to machine learning models and/or artificial intelligence and, more specifically, but not exclusively, to systems and methods for training machine learning models and/or for using machine learning models for selecting subjects for treatment and/or evaluation.

Traditionally, patients are prioritized for treatment and/or evaluation to reduce and/or prevent clinical outcomes (e.g., heart attack, exacerbation of chronic obstructive pulmonary disease (COPD), hospitalization) by a best manual guess, and/or treated reactively, with emergencies and/or sudden deteriorations being prioritized.

### SUMMARY

According to a first aspect, a computer implemented method of training a plurality of machine learning models and providing a combined prioritization component for dynamic prioritization of subjects for evaluation and/or treatment, comprises: accessing electronic medical records (EMR) of a set of a plurality of subjects, for each of a plurality of specific medical interventions that are resource limited and/or for each respective target clinical outcome: accessing a respective specific priority list of a respective sub-set of the plurality of subjects scheduled in a prioritized sequence for treatment and/or evaluation for the respective target clinical outcome and/or by the respective specific medical intervention, creating a respective specific training dataset that includes data extracted from the EMRs of at least the respective sub-set of the plurality of subjects labelled with the specific priority list, and training a respective specific machine learning model on the respective specific training dataset for generating an outcome of a respective target specific priority list of a sub-set of target subjects for prioritized evaluation and/or treatment for the respective target clinical outcome and/or by the respective specific medical intervention, in response to an input of data extracted from EMR of at least the sub-set of target subjects, accessing a combined prioritization component for generating an outcome of a target combined priority list of target subjects for prioritized evaluation and/or treatment in response to an input of outcomes of the plurality of specific machine learning models, and providing the plurality of specific machine learning models and the combined prioritization component.

According to a second aspect, a computer implemented method of training a plurality of machine learning models and providing a combined prioritization component for dynamic prioritization of target subjects for at least one of evaluation, and treatment, comprising: accessing electronic medical records (EMR) of a set of a plurality of subjects, for at least one of each of a plurality of specific medical interventions that are resource limited, and for each respective target clinical outcome: accessing a respective specific priority list of a respective sub-set of the plurality of subjects scheduled in a prioritized sequence for at least one of treatment and evaluation for at least one of the respective target clinical outcome and by the respective specific medical intervention, creating a respective specific training dataset that includes data extracted from the EMRs of at least the respective sub-set of the plurality of subjects labelled with the specific priority list, and training a respective specific machine learning model on the respective specific training dataset for generating an outcome of a respective target specific priority list of a sub-set of target subjects for prioritized at least one of evaluation and treatment for the at least one of respective target clinical outcome and by the respective specific medical intervention, in response to an input of data extracted from EMR of at least the sub-set of target subjects, accessing an intermediate component for computing a plurality of weighted specific priority lists by assigning a respective preselected global list weight to each respective target specific priority list outcome of respective specific machine learning models, wherein the same respective preselected global list weight is applied to all scores of all subjects of the same respective target specificity priority list, wherein each respective weight of each respective priority list is adjustable, accessing a combined prioritization component for generating an outcome of a target combined priority list of target subjects for prioritized at least one of evaluation and treatment in response to an input of the plurality of weighted specific priority lists, and providing the plurality of specific machine learning models, the intermediate component, and the combined prioritization component, wherein the plurality of specific machine learning models and the combined prioritization component generate a respective numerical score for each respective subject, and the specific priority lists and the combined priority list are created by ranking subjects according to respective numerical scores, wherein the combined prioritization component computes, for each respective subject of the plurality of target subjects, a respective weighted score by multiplying each respective numerical score assigned to the respective subject on each respective priority list by the respective global list weight assigned to the respective list.

In a further implementation of the first or second aspect, respective preselected global list weights indicating the magnitude of prioritization of the respective target specific priority lists comprise a numerical value.

In a further implementation of the first or second aspect, respective preselected global list weights indicating the magnitude of prioritization of the respective target specific priority list are selected according to a geographical location. In some examples, a geographical location may be automatically detected by a system disclosed herein using location identifiers (e.g., via the IP address of the computerized device used by the user for accessing functionalities and/or modules of the system, by a location tag that may be associated with a subject and/or with each authorized user of the system, etc.).

In a further implementation of the first or second aspect, respective preselected global list weights indicating the magnitude of prioritization of the respective target specific priority list are defined according to a set of rules.

In a further implementation of the first or second aspect, respective preselected global list weights indicating the magnitude of prioritization of the respective target specific priority list are fixed within a defined range of values.

In a further implementation of the first or second aspect, respective preselected global list weights indicating the magnitude of prioritization of the respective target specific priority list are adjustable to different values by different users.

In a further implementation of the first or second aspect, the combined prioritization component comprises a machine learning model, and further comprising creating a main training dataset that includes a plurality of specific priority lists obtained as outcomes of a plurality of specific machine learning models, labelled with a respective combined priority list of the set of the plurality of subjects for treatment and/or evaluation, and training the combined prioritization component on the main training dataset.

In a further implementation of the first or second aspect, the combined prioritization component comprises aggregation code that when executed by a processor aggregates, for each subject of the plurality of subjects, the respective numerical scores of the plurality of specific priority lists into an aggregated score, and generates the target combined priority list by ranking the plurality of subjects according to respective aggregated scores.

In a further implementation of the first or second aspect, each of the plurality of priority lists is assigned a respective weight, and wherein aggregating comprises, computing, for each respective subject a respective weighted score by multiplying each respective numerical score assigned to the respective subject on each respective priority list by the respective weight assigned to the respective list, and ranking the plurality of subjects according to respective weighted scores, the combined priority list comprising the ranked weighted scores.

In a further implementation of the first or second aspect, at least one of a plurality of the target specific priority lists and the target combined priority list are time correlated priority lists, denoting, for each respective rank within each respective time correlated priority list, a maximal recommended time interval for performing a corresponding medical intervention on the respective subject at the respective rank for reducing or preventing a target clinical outcome.

In a further implementation of the first or second aspect, each respective specific training dataset and/or a main training dataset further includes a label of at least one of: occurrence of the target clinical outcome, an indication of the maximal recommended time interval, and a history of previous time intervals between respective medical interventions.

In a further implementation of the first or second aspect, at least one of a plurality of the target specific priority lists and the target combined priority list are time correlated priority lists, wherein each respective rank within each respective time correlated priority list denotes a recommended maximal time interval for performing a corresponding medical intervention on the respective subject for an effective allocation of resources for performing the medical intervention based on an input of a schedule of availability of resources for performing each respective corresponding medical intervention for each of a plurality of time intervals.

In a further implementation of the first or second aspect, each respective specific training dataset and/or a main training dataset further includes a schedule of availability of resources for performing each respective corresponding medical intervention for each of a plurality of time intervals, and the respective machine learning model is trained for generating a respective time correlated priority list in response to an input of a schedule of time correlated availability of the respective resource for performing the respective medical intervention.

In a further implementation of the first aspect, further comprising: defining an intermediate component that assigns a respective preselected weight to each respective specific priority list to compute a plurality of weighted specific priority lists, wherein the plurality of weighted specific priority lists are fed into the combined prioritization component.

In a further implementation of the first or second aspect, the respective specific training dataset further includes, for each respective sub-set of the plurality of subjects, an indication of risk of a respective clinical outcome and/or a diagnosis of a respective clinical diagnosis, wherein each respective specific machine learning model is for a respective clinical outcome and/or for the respective clinical diagnosis, wherein each respective specific machine learning model is trained to generate the outcome of the respective target priority list of the sub-set of target subjects at risk for the clinical outcome and/or diagnosed with the respective clinical diagnosis, for prioritized evaluation and/or treatment for the respective clinical outcome and/or respective clinical diagnosis and/or target clinical outcome by the respective specific medical intervention.

In a further implementation of the first or second aspect, further comprising: receiving a set of target EMRs of a plurality of target subjects, feeding the set of target EMRs into the plurality of specific machine learning models, obtaining a plurality of specific priority lists as outcomes of the plurality of specific machine learning models, feeding the plurality of specific priority lists into the combined prioritization component, and obtaining a combined priority list as an outcome of the combined prioritization component.

In a further implementation of the first or second aspect, further comprising: sequentially treating and/or evaluating the plurality of target subjects for a plurality of respective target clinical outcomes by the plurality of specific medical interventions according to a prioritized order defined by the plurality of specific priority lists, and sequentially treating and/or evaluating the plurality of target subjects according to the prioritized order defined by the combined priority list.

In a further implementation of the first or second aspect, further comprising monitoring the set of target EMRs for detecting at least one of: updated EMR including change in at least one value of at least one field, degradation of performance of one or more of the ML models, change in infrastructure demand, and or user request, and in response to a detected change, feeding the at least one updated EMR into the plurality of specific machine learning models for obtaining an updated plurality of specific priority lists, and feeding the updated plurality of specific priority lists into the combined prioritization component for obtaining an updated combined priority list.

In a further implementation of the first or second aspect, the plurality of specific machine learning models and/or the combined prioritization component include at least a recurrent neural network (RNN) component and/or at least a graph neural network (GNN) component.

In a further implementation of the first or second aspect, the plurality of specific medical interventions are selected from a group consisting of: surgical procedure, catheterization procedure, laboratory testing, lifestyle modification, medical imaging, non-imaging work-up, cancer screening test, and medical specialist consultation.

In a further implementation of the first or second aspect, further comprising treating and/or evaluation target subjects by a main medical intervention according to the target combined priority list.

In a further implementation of the first or second aspect, the main medical intervention comprises a medical provider consultation.

In a further implementation of the first or second aspect, the plurality of specific machine learning models and the combined prioritization component generate the specific priority lists and the combined priority list as a respective single simultaneous outcome, wherein a ranking within a respective list is relative to other subjects within the list.

In a further implementation of the first aspect, the plurality of specific machine learning models and the combined prioritization component generate a respective numerical score for each respective subject, and the specific priority lists and the combined priority list are created by ranking subjects according to respective numerical scores.

In a further implementation of the first or second aspect, further comprising monitoring the specific priority lists and the combined priority list for changes in ranking of subjects therein, and in response to a detected change, updating the respective specific training dataset and/or a main training dataset, and updating the training of the specific machine learning models and/or the combined prioritization component.

In a further implementation of the first or second aspect, further comprising outputting for each respective subject, at least one of: the respective numerical score computed by the combined prioritization component, and a ranking of the respective subject on the combined priority list.

In a further implementation of the first or second aspect, wherein each respective weight of each respective priority list is dynamically adjustable by a user via a user interface.

In a further implementation of the first or second aspect, the user interface comprises a graphical user interface (GUI) presented on a display.

According to a third aspect, a computer implemented method for prioritization of subjects for treatment and/or evaluation, comprises: accessing EMRs of a set of a plurality of subjects, feeding the EMRs into each of a plurality of specific machine learning models, obtaining a plurality of specific priority lists as outcomes of the plurality of specific machine learning models, wherein each respective specific priority list includes a respective sub-set of the plurality of subjects scheduled in a prioritized sequence for treatment and/or evaluation for a respective target clinical outcome and/or by a respective specific medical intervention, feeding the plurality of specific priority lists into combined prioritization component, and obtaining a combined priority list as an outcome of the combined prioritization component, wherein the combined priority list is of the plurality of subjects indicating priority for evaluation and/or treatment.

According to a fourth aspect, A computer implemented method for prioritization of target subjects for at least one of treatment and evaluation, comprising accessing EMRs of a set of a plurality of subjects, feeding the EMRs into each of a plurality of specific machine learning models, obtaining a plurality of specific priority lists as outcomes of the plurality of specific machine learning models, wherein each respective specific priority list includes a respective sub-set of the plurality of subjects scheduled in a prioritized sequence for at least one of treatment and evaluation for at least one of a respective target clinical outcome and by a respective specific medical intervention, feeding the plurality of specific priority lists into an intermediate component that assigns a respective preselected global list weight to each respective target specific priority list, wherein the same respective preselected global list weight is applied to all scores of all subjects of the same respective target specificity priority list, wherein each respective preselected weight of each respective priority list is adjustable, obtaining a plurality of weighted specific priority lists from the intermediate component, feeding the plurality of weighted specific priority lists into combined prioritization component, and obtaining a combined priority list as an outcome of the combined prioritization component, wherein the combined priority list is of the plurality of subjects indicating priority for at least one of evaluation and treatment, wherein the plurality of specific machine learning models and the combined prioritization component generate a respective numerical score for each respective subject, and the specific priority lists and the combined priority list are created by ranking subjects according to respective numerical scores, wherein the combined prioritization component computes, for each respective subject of the plurality of target subjects, a respective weighted score by multiplying each respective numerical score assigned to the respective subject on each respective priority list by the respective global list weight assigned to the respective list.

According to a fifth aspect, a computer implemented method for dynamic prioritization of subjects for evaluation and/or treatment, comprises: accessing a plurality of specific priority lists of a respective sub-set of a plurality of subjects scheduled in a prioritized sequence for treatment and/or evaluation for a respective target clinical outcome and/or by a respective specific medical intervention, wherein each subject in each of the plurality of specific priority lists is associated with a respective numerical score indicative of risk of a respective clinical outcome, aggregating, for each subject of the plurality of subjects, the respective numerical scores of the plurality of specific priority lists into an aggregated score, and generating a combined priority list by ranking the plurality of subjects according to respective aggregated scores.

According to a sixth aspect, a computer implemented method for dynamic prioritization of target subjects for at least one of evaluation and treatment, comprises: accessing a plurality of specific priority lists of a respective sub-set of a plurality of subjects scheduled in a prioritized sequence for at least one of treatment and evaluation for at least one of a respective target clinical outcome and by a respective specific medical intervention, wherein each subject in each of the plurality of specific priority lists is associated with a respective numerical score indicative of risk of a respective clinical outcome, wherein each of the plurality of priority lists is assigned a respective global list weight that is adjustable, wherein the same respective preselected global list weight is applied to all scores of all subjects of the same respective target specificity priority list, computing, for each respective subject of the plurality of subjects, a respective weighted score by multiplying each respective numerical score assigned to the respective subject on each respective priority list by the respective global list weight assigned to the respective list, generating a combined priority list by ranking the plurality of subjects according to respective weighted scores, wherein the combined priority list comprising the ranked weighted scores.

In a further implementation of the fifth or sixth aspect, further comprising: computing each of the plurality of specific priority lists for each respective sub-set of the plurality of subjects according to data extracted from EMRs of the respective sub-set of the plurality of subjects.

In a further implementation of the fifth or sixth aspect, at least one specific priority list is computed by applying a set of rules to the data extracted from the EMR of the respective sub-set of the plurality of subjects.

In a further implementation of the fifth or sixth aspect, further comprising: creating at least one specific training dataset that includes data extracted from EMRs of at least one respective sub-set of the plurality of subjects, wherein data of each subject is labelled with a respective numerical score of the respective combined priority list, training at least one specific machine learning model, each respective specific machine learning model trained on a respective specific training dataset, wherein at least one specific priority list is computed as an outcome of a respective trained specific machine learning model in response to an input of data extracted from EMRs of the respective sub-set of the plurality of subjects.

In a further implementation of the fifth aspect, each of the plurality of priority lists is assigned a respective weight, and wherein aggregating comprises, computing, for each respective subject a respective weighted score by multiplying each respective numerical score assigned to the respective subject on each respective priority list by the respective weight assigned to the respective list, and ranking the plurality of subjects according to respective weighted scores, the combined priority list comprising the ranked weighted scores.

In a further implementation of the fifth aspect, each respective weight of each respective priority list is dynamically adjustable by a user via a graphical user interface (GUI) presented on a display.

In a further implementation of the fifth or sixth aspect, further comprising: sequentially treating and/or evaluating the plurality of subjects for a plurality of target clinical outcomes and/or by a plurality of specific medical interventions according to a prioritized order defined by the plurality of specific priority lists, and sequentially treating and/or evaluating the plurality of subjects according to the prioritized order defined by the combined priority list.

In a further implementation of the fifth or sixth aspect, the plurality of specific priority lists and the combined priority list are each arranged as a respective ordered ranking of the respective sub-set of the plurality of subjects, wherein a certain ranking within a respective list is relative to other subjects within the respective list.

In a further implementation of the fifth or sixth aspect, the plurality of specific priority lists and the combined priority list each store a respective numerical score for each respective subject, and the specific priority lists and the combined priority list are created by ranking subjects according to respective numerical scores.

According to a seventh aspect, a computer implemented method of scheduling timeslots for evaluation and/or treatment by, comprises: accessing at least one risk parameter for each subject of a plurality of subjects, the at least one risk parameter indicative of risk of a certain clinical outcome and/or a diagnosis of a certain clinical diagnosis, and mapping, for each respective subject, the at least one risk parameter to a certain maximal recommended time interval for performing a corresponding medical intervention on the respective subject for reducing or preventing a target clinical outcome.

According to an eighth aspect, a computer implemented method of scheduling timeslots for at least one of evaluation and treatment by, comprises: accessing at least one risk parameter for each subject of a plurality of subjects, the at least one risk parameter indicative of risk of at least one of a certain clinical outcome and a diagnosis of a certain clinical diagnosis, mapping, for each respective subject, the at least one risk parameter to a certain maximal recommended time interval for performing a corresponding medical intervention on the respective subject for reducing or preventing a target clinical outcome, wherein the certain maximal recommended time interval comprises a frequency for at least one of evaluation and treatment at temporal intervals that do not exceed the certain maximal recommended time interval, and presenting within a GUI presented on a display, candidate timeslots for at least one of evaluation and treatment that have dates falling within and not exceeding the certain maximal recommended time interval.

In a further implementation of the seventh or eighth aspect, the at least one risk parameter comprises a risk score in a defined range, and mapping comprises mapping risk scores in each of a plurality of sub-ranges of the defined range to respective maximal recommended time intervals.

In a further implementation of the seventh aspect, further comprising: presenting within a GUI presented on a display, candidate timeslots for evaluation and/or treatment that have dates falling within and not exceeding the certain maximal recommended time interval.

In a further implementation of the fourth aspect, the certain maximal recommended time interval comprises a frequency for evaluation and/or treatment at temporal intervals that do not exceed the certain maximal recommended time interval.

In a further implementation of the seventh aspect, the mapping is performed based on a set of rules that are dynamically adjustable by a user via a GUI presented on a display and/or prefixed.

In a further implementation of the seventh or eighth aspect, the mapping is performed by feeding a plurality of subject parameters extracted from an EMR of the respective subject, into a trained mapping machine learning model that generates an outcome of the certain maximal recommended time interval, wherein the at least one risk parameter is represented by internal embeddings of the machine learning model, wherein the machine learning model is trained on a training dataset of sample sets of subject parameters extracted from the EMR of each of a plurality of sample subjects, each sample set labelled with a ground truth indication of a respective maximal recommended time interval.

In a further implementation of the seventh or eighth aspect, further comprising: setting an appointment, for a respective subject, for performing the corresponding medical intervention at a date falling within and not exceeding the certain maximal recommended time interval, determining that the respective subject did not attend the appointment, and rescheduling another appointment for performing the corresponding medical intervention at a date falling within and not exceeding the certain maximal recommended time interval.

In a further implementation of the seventh or eighth aspect, the corresponding medical intervention comprises a review of the EMR of the respective subject by a healthcare provider without the presence of the subject, and further comprising after the healthcare provider performs the review of the EMR of the respective subject: computing an update of the at least one risk parameter for the respective subject, performing an updated mapping of the update of the at least one risk parameter to an updated maximal recommended time interval for performing another of the corresponding medical intervention, presenting within a GUI, at least one future timeslot for performing another review of an updated EMR of the respective subject, wherein the at least one future timeslots fall within and do not exceed the certain maximal recommended time interval from a date of the previously performed review of the EMR of the respective subject, and scheduling the future timeslot in response to a selection of one of the at least one future timeslot.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of a system for computing a combined priority list from multiple specific priority lists each computed from data extracted from EMRs, and/or computing maximal recommend time intervals for performing a respective medical intervention, in accordance with some embodiments of the present invention;

FIG. 2 is a flowchart of a method of prioritization of subjects for treatment and/or evaluation, in accordance with some embodiments of the present invention;

FIG. 3 is a flowchart of a method of training multiple machine learning models and/or a combined prioritization component for dynamic prioritization of subjects for evaluation and/or treatment, in accordance with some embodiments of the present invention;

FIG. 4 is a flowchart of a method of computing maximal recommended time intervals for evaluation and/or treatment of subjects, in accordance with some embodiments of the present invention; and

FIG. 5 is a schematic of a GUI presenting examples of specific priority lists, in accordance with some embodiments of the present invention;

FIG. 6 is a schematic of a GUI as shown in FIG. 5, with a further horizontal scroll to the right, displaying additional data for patients on the selected specific priority list, in accordance with some embodiments of the present invention;

FIG. 7 is a schematic of a GUI presenting an exemplary combined priority list, in accordance with some embodiments of the present invention;

FIG. 8 is a schematic of the GUI of FIG. 7 expanded for “Patient B”, presenting multiple specific priority lists that include Patient B, which are combined and/or aggregated to determine the ranking of Patient B on the combined priority list, in accordance with some embodiments of the present invention;

FIG. 9 is a schematic of a GUI presenting a maximal recommended time interval for performing a review of the clinical file of “Patient A” for reducing or preventing the target clinical outcome of “Deterioration from chronic illness”, in accordance with some embodiments of the present invention; and

FIG. 10 is a schematic of a GUI presenting a maximal recommended time interval for performing a review of the clinical file of “Patient B” for reducing or preventing the target clinical outcome of “Deterioration from chronic illness”, in accordance with some embodiments of the present invention.

### DETAILED DESCRIPTION

The present invention, in some embodiments thereof, relates to machine learning models and/or artificial intelligence and, more specifically, but not exclusively, to systems and methods for training machine learning models and/or for using machine learning models for selecting subjects for treatment and/or evaluation.

An aspect of some embodiments of the present invention relates to systems, methods, an apparatus, and/or code instructions (stored on a memory and executable by hardware processors) for dynamic prioritization of subjects for evaluation and/or treatment. Multiple specific priority lists are computed and/or accessed. Each respective priority list is of a respective sub-set of subjects which are scheduled in a respective prioritized sequence for treatment and/or evaluation by a respective set of relevant specific medical interventions, for example, medication prescription, cardiac catheterization, laboratory work, assessment by a clinician, lifestyle modifications, and specialist consultation. The subjects within each respective priority list are ordered, where the ordering defines a ranking of priority, i.e., subjects higher on the list may represent subjects that should be treated and/or evaluated first, for example, since they are at higher risk of developing a clinical outcome and/or since they are most likely to benefit from the intervention (e.g., reduction in risk of developing the clinical outcome, and/or they have the clinical outcome and are most likely to benefit). The priority lists may be implemented, for example, as a linked list, and/or an ordering of numerical scores associated with each subject. Each subject in each of the specific priority lists may be associated with a respective numerical score indicative of risk of a respective clinical outcome, for example, likelihood of stroke in the next 30 days, or likelihood of hospitalization. Alternatively or additionally, the numerical score is indicative of likelihood of improving in response to the respective intervention. The specific priority lists may be outcomes of respective trained machine learning (ML) models and/or one or more sets of rules, that are fed data extracted from electronic health/medical records of the subjects. A combined priority list is generated by aggregating the subjects of the multiple specific priority lists, into a single ordered list, where the order of the subjects of the combined priority list represents an ordering for treatment and/or evaluation of the subjects. Subjects higher up on the combined priority list are prioritized for treatment and/or evaluation, for example, due to higher risk of clinical outcomes and/or being most likely to benefit from the intervention, for example, most likely to be cured, experience an increase in quality of life, return to work, be discharged from hospital, and/or perform independently at home. Respective numerical scores of subjects on the specific priority lists may be aggregated into an aggregated score. A combined priority list may be generated by ranking the subjects according to respective aggregated scores. Each respective priority list may be associated with a selected global list weight that indicates a magnitude of prioritization of the respective target specific priority list, for example, a numerical value within a range, where the numerical value indicates the magnitude of prioritization. The global list weights may be, for example, dynamically adjusted, preselected, adjusted differently by different users, bound within defined ranges, selected according to geographical locations, defined by a set of rules, and the like. The global list weights may be presented and/or dynamically selected by a user (e.g. via a graphical user interface (GUI)). A respective weighted score may be computed for each subject, by multiplying each respective numerical score assigned to the respective subject on each respective priority list by the respective global list weight assigned to the respective list. The same global list weight is applied to each subject of the corresponding specific priority list. The combined priority list may be created by ranking the weighted scores of the subjects. The combined priority list includes all the subjects which appear on different lists, i.e., each subject appears on at least one of the specific priority lists. The combined priority list may represent a holistic view of ranking of the most urgent cases to be seen by a clinician, based on the ranking of each patient on the different priority lists. This may provide busy clinicians with the ability to treat and/or evaluate the most urgent cases in a proactive manner. Alternatively or additionally, the combined priority list may represent a holistic view of ranking of the subjects more likely to benefit upon intervention, for example, most likely to be cured, experience an increase in quality of life, return to work, be discharged from hospital, and/or perform independently at home. Clinical prioritization may not be obvious to clinicians having to decide between patients suffering from different sets of clinical conditions.

The subjects on the combined priority list may be prioritized for evaluation and/or treatment, optionally by a main medical intervention that is resource limited. The main medical intervention and/or end user may represent one or more of the following:
An end user with access to view multiple specific priority lists of subsets of subjects (e.g., patients) and/or to view a combined priority list of the subjects (e.g., all patients appearing in all lists, and/or most highly ranked patients) generated by combining the multiple specific priority lists.
A human that performs an evaluation and/or treatment of the subjects, for example, an offline review of the respective subject's files, and/or a consult with the respective subject (e.g., in person, phone, telemedicine). Examples of human main medical interventions and/or end users include, a family physician, general internist, general practitioner, other medical specialist (e.g., cardiologist, radiologist, surgeon), nurse practitioner, physician assistant, pediatrician, primary care provider, social worker, physiotherapist, nutritionist, and/or other person responsible for multiple patients with limited time availability.
The main medical intervention may be a medical procedure and/or evaluation performed automatically by a device and/or manually by a human and/or semi-manually such as a human operating a device. For example, PET/CT scan, cardiac catheterization procedure, surgical procedure (e.g., knee implant), and lab work.
The main medical intervention may be one or a combination of specific medical interventions, as described below. For example, the main medical intervention may be a cardiac catheterization procedure, where the combined priority list (e.g., as described herein) is a ranking of subjects for priority cardiac catheterization, based on specific priority lists of specific medical interventions as described below (e.g., a first list of patients with severe diabetes, a second list of patients at highest risk of heart attack, etc.).
The main medical intervention may be resource limited, for example, in terms of time (e.g., available a certain number of hours a day) and/or in terms of rate of treatment and/or evaluation of subjects per unit time (e.g., able to evaluate and/or treat 10 patients per hour). For example, a family physician may be able to schedule a maximum of 10 patients per hour, for 8 hours a day. In another example, an imaging facility with 3 CT scanners may be able to handle 6 patients per hour, for 12 hours a day.

An aspect of some embodiments of the present invention relates to systems, methods, an apparatus, and/or code instructions (stored on a memory and executable by hardware processors) for scheduling timeslots for evaluation and/or treatment, optionally by a main medical intervention that is resource limited, for example, by nurses, physician assistants, and/or primary care physicians and other clinicians. One or more risk parameters are obtained for each subject of multiple subjects, for example, for each patient of the group of patients. Each risk parameter indicates risk of a certain clinical outcome and/or diagnosis of a certain clinical diagnosis. For example, risk of stroke in the next 12 months, risk of hospitalization in the next 30 days, risk of developing type 2 diabetes, risk of non-compliance with medication, and risk of chronic obstructive pulmonary disease (COPD) exacerbation. The risk parameter (e.g., value) may indicate for example, absolute risk scores or relative scores such as risk percentiles. For each respective subject, the risk parameter(s) are mapped to a certain maximal recommend time interval for performing a corresponding medical intervention on the respective subject at a respective rank for reducing and/or preventing the target clinical outcome. For example, a risk parameter for stroke in the range of 0.8-1.0 indicates high risk, and is mapped to a maximal recommended time interval of 30 days for being treated and/or evaluated to reduce or prevent stroke. The certain maximal recommended time interval may be a frequency for evaluation and/or treatment at temporal intervals that each do not exceed the maximal recommended time interval. For example, maximum time between follow-up appointments is 6 months.

An aspect of some embodiments of the present invention relates to systems, methods, an apparatus, and/or code instructions (stored on a memory and executable by hardware processors) for training machine learning models and providing and/or training a combined prioritization component, for dynamic prioritization of subjects for evaluation and/or treatment (e.g., by the main medical intervention that is resource limited). Electronic medical/health records (EMR) of a set of subjects is accessed. A respective specific priority list indicating a respective target clinical outcome of a respective sub-set of the subjects sorted in a prioritized sequence for treatment and/or evaluation is accessed. The respective specific priority list may indicate ranked risk of the respective subject developing the respective target clinical outcome. Examples of target clinical outcomes include: heart attack, exacerbation of chronic obstructive pulmonary disease (COPD), and hospitalization. Alternatively or additionally, for each specific medical intervention that is resource limited (e.g., PET/CT scan, cardiac catheterization procedure, lab work, assessment by a clinician, lifestyle modification, and/or specialist consult), a respective specific priority list of a respective sub-set of the subjects scheduled in a prioritized sequence for treatment and/or evaluation by the respective specific medical intervention, is accessed. The specific medical intervention may be for treatment and/or evaluation of the respective target clinical outcome. A respective specific training dataset that includes data extracted from the EMRs of at least the respective sub-set of subjects may be labeled with the respective specific priority list. A respective specific machine learning model is trained on the respective specific training dataset for generating an outcome of a respective target specific priority list of a sub-set of target subjects for prioritized evaluation and/or treatment for the respective specific clinical outcome, optionally by the respective specific medical intervention, in response to an input of data extracted from EMR of at least the sub-set of target subjects. Alternatively or additionally, the respective specific machine learning model is trained on the respective specific training dataset for generating an outcome of a respective target specific priority list of a sub-set of target subjects for prioritized evaluation and/or by the respective specific medical intervention, optionally for the respective target clinical outcome, in response to an input of data extracted from EMR of at least the sub-set of target subjects. A combined prioritization component is accessed. The combined prioritization component generates an outcome of a target combined priority list of target subjects for prioritized evaluation and/or treatment in response to an input of outcomes of the specific machine learning models. The specific machine learning models and the combined prioritization component are provided.

Optionally, the combined prioritization component is implemented as a machine learning model. A main training dataset that includes specific priority lists obtained as outcomes of the specific machine learning models, labeled with a respective combined priority list of the set of the subjects for treatment and/or evaluation, is created. The combined prioritization component is trained on the main training dataset.

Alternatively or additionally, the combined prioritization component is implemented as code that when executed by a processor aggregates, for each subject, respective numerical scores of the specific priority lists into an aggregated score, and generates the target combined priority list by ranking the subjects according to respective aggregated scores. Each of the priority lists is assigned a respective weight. A respective weighted score is computed for each respective subject by multiplying each respective numerical score assigned to the respective subject on each respective priority list by a respective weight assigned to the respective list. The subjects are ranked according to respective weighted scores. The combined priority list is created from the order of the ranked weighted scores.

An aspect of some embodiments of the present invention relates to systems, methods, an apparatus, and/or code instructions (stored on a memory and executable by hardware processors) for prioritization of subjects for treatment and/or evaluation (e.g., by the main medical intervention that is resource limited). EMRs of a set of subjects are accessed, for example, patients of a certain primary care physician. The EMRs (and/or features extracted from the EMRs) are fed into each respective specific machine learning models. It is noted that the data and/or features may not necessarily be extracted directly from the EMR, but may be extracted from another data source that documents data of subjects, for example, a central data warehouse, and/or other sources of individualized medical information, for example, imaging systems, laboratory systems, and pharmacy purchases. Specific priority lists as obtained as outcomes of the specific machine learning models. Each respective specific priority list includes a respective sub-set of the subjects scheduled in a prioritized sequence for treatment and/or evaluation by a respective specific medical intervention. The specific priority lists are fed into a combined prioritization component. A combined priority list is obtained as an outcome of the combined prioritization component. The combined priority list is of the subjects indicating priority for evaluation and/or treatment. The subjects may be evaluated and/or treated according to the combined priority list.

At least some implementations of the systems, methods, apparatus, and/or code instructions described herein address the technical problem of customization of prioritization of specific priority lists obtained as outcomes of machine learning models, for combination into a combined priority list. At least some implementations of the systems, methods, apparatus, and/or code instructions described herein improve the technical field of machine learning models, by providing an approach for customization of prioritization of specific priority lists obtained as outcomes of machine learning models, for combination into a combined priority list. At least some implementations of the systems, methods, apparatus, and/or code instructions described herein improve over existing approaches for prioritization of outcomes of machine learning models. In at least some embodiments, the solution to the technical problem, the improvement to the technical field, and/or the improvement over prior approaches, are based on assignment of global list weights to the specific priority lists (i.e., a respective global list weight per specific priority list). The global list weight is applied to numerical values assigned to each subject of the corresponding specific priority list, which are used to computed weighted values used to create the combined priority list. The global list weight indicates a magnitude of prioritization of the respective target specific priority list. The magnitude may be an absolute value, for example, the global list weight of a first specific priority list is 2.5 and the global list weight of a second specific priority list is 3.6. The magnitude may be a relative value, for example, the global list weight of a first specific priority list is 2× the global list weight of a second specific priority list. The global list weight may be implemented as a numerical value within a range, where the numerical value indicates the magnitude of prioritization. The global list weights may be, for example, dynamically adjusted, preselected, adjusted differently by different users, bound within defined ranges, selected according to geographical locations, defined by a set of rules, and the like. The global list weight may provide a fine (e.g., high resolution) magnitude of prioritization, for example, as discrete values, numbers with one, two, three or more decimals, and the like.

The global list weights enables customization of the combined priority list for different uses. For examples, in one healthcare clinic where immunization rates against coronavirus (COVID-19) is low, the global list weight for the coronavirus immunization specific priority list may be assigned at a higher value than a second healthcare clinic where immunization rates against coronavirus is high (i.e., to prioritize non-vaccinated individuals). In yet another example, in one clinic where there is a long waiting list for MRI scans, the global list weight for the MRI scan specific priority list may be assigned at a higher value than a second healthcare clinic where MRI scan wait times are low (i.e., to prioritize those that need MRI scans the most, to get them quicker).

The magnitude of prioritization is different than other approaches where prioritization may be mere relative, for example, the list outputted by a first ML model is prioritized over the list outputted by a second ML model, which provides no indication of magnitude. In such prior approaches, two users that both prioritize the first ML model over the second ML model for the same set of specific priority lists, would obtain the same combined priority list. In contrast, in accordance with at least some embodiments described herein, the magnitude of prioritization by the global list weights may be set, such that even if two users both prioritize the first ML model over the second ML model for the same set of specific priority lists, the magnitude of the global list weights result in two different combined priority lists being generated.

An example is now provided to help understand how the magnitude of the global list weights may result in two different combined priority lists being generated, even when the relative prioritization (e.g., model 1 is prioritized over model 2) is maintained. Consider two general practice (GP) clinics working in different locations. Both use two specific priority lists of patients, for cardiovascular disease (CVD) and for osteoporosis. Both doctors agree that the CVD specific priority list is to be assigned a higher prioritization than the osteoporosis specific priority list, for example, since CVD is associated with more morbidity and mortality overall, but to a different extent. One doctor thinks CVD patients with even fairly low risk should appear higher in the combined priority list than high risk osteoporosis patients, and as such assigns the CVD specific priority list a global list weight of 5 and assigns the osteoporosis specific priority list a global list weight of 1. In this first case, individuals with a risk percentile higher than 20 (i.e., the top 80% of the CVD list) will appear above someone in the top percentile of the osteoporosis list. I.e. in the first combined list generated using the respective global list weights of 5 and 1, high risk osteoporosis patients will almost entirely appear lower down than the CVD patients. Now, the other doctor thinks that patients on the CVD list should be slightly relatively higher up in the combined priority list than the equivalent risk of osteoporosis, and decides to use a global list weight of 1.5 for the CVD list, and the same global list weight of 1 for the osteoporosis list. In this second example, all individuals with a risk percentile greater than 67 will appear in the combined priority list above individuals in the top percentile of the osteoporosis list. I.e., in the second combined priority list generated using the respective global list weights of 1.5 and 1, high risk osteoporosis patients will still appear reasonably high up the combined priority list and will be intertwined with the moderate risk CVD patients. Therefore, even though in both cases CVD is prioritized over osteoporosis, the different global list weights results in different combined priority lists using the same set of specific CVD and osteoporosis priority lists. In contrast, in approaches there prioritization is relative without magnitude, the same combined priority lists will be generated for both cases where CVD is relatively prioritized over osteoporosis (i.e., without magnitude) using the same set of specific CVD and osteoporosis priority lists. It is noted that some individuals may be at risk of both diseases, which could make the example more complicated. However, either way, the combined priority list ranking will be given according to the users preferences, which may be clinically based (e.g., each individual may decide on which specific priority lists they would prefer to focus on according to their clinical judgement).

Referring now to Tables 1 and 2, a computational example is provided. Tables 1 represents computations by a first user, and table 2 represents computations by a second user. Both users wish to prioritize CVD patients, but differ in how they wish to prioritize osteoporosis patients. User 1 wishes to assign a lower priority to the osteoporosis patients, and assigns a global weight value of 0.3. User 2 wishes to assign a higher priority to the osteoporosis patients, and assigns a global weight value of 0.5. Both users assign a global weight value of 0.7 to the CVD patients. In both Tables 1 and 2, the scores on the specific CVD list, and the scores on the specific osteoporosis list are the same for all subjects, since the same specific priority lists are used.

The difference in magnitude of the osteoporosis global list weight creates a different relative value of the combined scores computed for the subjects, which results in different rankings in the combined lists for user 1 and user 2. According to Table 1, John is highest ranked with a combined score of 16, then Jack with a combined score of 7.2, and finally Jane with a combined score of 6.7. In contrast, according to Table 2, John is highest ranked with a combined score of 22, however, then Jane is ranked second with a combined score of 10.7, and finally Jack is ranked last with a combined score of 9.2. Hence, the different users, by setting different magnitudes of the global list weights, even where the weights remain relative to each other, i.e., the osteoporosis global list weight is less than the CVD global list weight for both Tables 1 and 2, create different combined priority lists.

TABLE 1
Adjustable magnitude of global list weights - First user

| | | | | | | | Combined |
|---|---|---|---|---|---|---|---|
| | | | | Score | CVD | | priority |
| | Score on | osteoporosis | osteoporosis | on | global | CVD | list (sum |
| | osteoporosis | global list | weighted | CVC | list | weighted | weighted |
| Subject | list | weight | score | list | weight | score | scores) |
| John | 30 | .3 | 9 | 10 | .7 | 7 | 16 |
| Jane | 20 | .3 | 6 | 1 | .7 | .7 | 6.7 |
| Jack | 10 | .3 | 3 | 6 | .7 | 4.2 | 7.2 |

TABLE 2
Adjustable magnitude of global list weights - Second user

| | | | | | | | Combined |
|---|---|---|---|---|---|---|---|
| | | | | Score | CVD | | priority |
| | Score on | osteoporosis | osteoporosis | on | global | CVD | list (sum |
| | osteoporosis | global list | weighted | CVD | list | weighted | weighted |
| Subject | list | weight | score | list | weight | score | scores) |
| John | 30 | .5 | 15 | 10 | .7 | 7 | 22 |
| Jane | 20 | .5 | 10 | 1 | .7 | .7 | 10.7 |
| Jack | 10 | .5 | 5 | 6 | .7 | 4.2 | 9.2 |

In contrast to Tables 1 and 2 which are based on global list weights that indicate magnitude, such as magnitude of relative prioritization, reference is now made to Tables 3 and 4 which are based on approaches of relative prioritization without indication of magnitude. In such other approaches, only relative priority of the different lists is considered without magnitude. For example, where global list weights are pre-fixed without indicating relative magnitude. Using the example described above for Tables 1 and 2, for both Tables 3 and 4, both users prioritize CVD over osteoporosis, which may refer, for example, to a fixed predefined weight for CVD of 3 and of osteoporosis of 1, without ability to select and/or define magnitude. As seen in Tables 3 and 4, since there is no feature to indicate magnitude of the weights, but only to indicate relative priority, the combined lists for Tables 3 and 4 are the same. I.e., in both lists, John is the highest priority, followed by Jack and then Jane. Other approaches do not provide the finer level of customization for different users that are provided by the global list weights described herein, resulting in different users having the same lists.

TABLE 3
Relative prioritization without magnitude - First user

| | | | | | | | Combined |
|---|---|---|---|---|---|---|---|
| | | | | Score | CVD | | priority |
| | Score on | osteoporosis | osteoporosis | on | global | CVD | list (sum |
| | osteoporosis | global list | weighted | CVD | list | weighted | weighted |
| Subject | list | weight | score | list | weight | score | scores) |
| John | 30 | 1 | 30 | 10 | 3 | 30 | 60 |
| Jane | 20 | 1 | 20 | 1 | 3 | 3 | 23 |
| Jack | 10 | 1 | 10 | 6 | 3 | 18 | 28 |

TABLE 4
Relative prioritization without magnitude - Second user

| | | | | | | | Combined |
|---|---|---|---|---|---|---|---|
| | | | | Score | CVD | | priority |
| | Score on | osteoporosis | osteoporosis | on | global | CVD | list (sum |
| | osteoporosis | global list | weighted | CVD | list | weighted | weighted |
| Subject | list | weight | score | list | weight | score | scores) |
| John | 30 | 1 | 30 | 10 | 3 | 30 | 60 |
| Jane | 20 | 1 | 20 | 1 | 3 | 3 | 23 |
| Jack | 10 | 1 | 10 | 6 | 3 | 18 | 28 |

Other improvement of at least some embodiments described herein relate to training dataset. Other prior approaches are based on training different sets of ML models on training datasets with different mixes of training data, to create ML models most suitable for different users. For example, for a first user that is a cardiologist, a first set of ML models is trained on a first set of training data that includes records of cardiac patients, and for a second user that is an orthopedic surgeon, a second set of ML models is trained on a second set of training data that includes records of orthopedic patients. Training different ML models on different training data for different users is time consuming, computationally inefficient, and difficult, for example, to obtain enough data for creating different training dataset, to create the different training dataset, and to train ML models on the different training datasets. In contrast, embodiments describes herein assign different sets of global list weights (e.g., for different users) to the same set of priority lists outputted by the same set of ML models, to obtain different combined priority lists. The different combined priority lists are obtained by adjustment of the global list weights, rather than by repeating the process of creating different training datasets and training the different ML models.

At least some implementations of the systems, methods, apparatus, and/or code instructions described herein address the technical problem of allocation of limited medical resources for medical intervention (e.g., medical treatment and/or evaluation) of subjects. Many medical resources used to treat and/or evaluate subjects are in limited supply, for example, imaging equipment (e.g., CT, MRI, PET/CT), cancer screening services (e.g., mammography, colonoscopy), surgical procedures, catheterization procedures, laboratory testing, medical consultations, and/or main medical interventions (e.g., time of primary care physicians, family doctors, nurses, physician assistants, nurse practitioners, pediatricians, and internists, and may optionally include and/or be designed for other sectors of health care workers such as social workers, pharmacists, physiotherapists, speech therapists, dentist, and/or auxiliary health providers) and the like. Subjects are at different risks for a clinical outcome (e.g., heart attack, stroke, death, COPD exacerbation, hospitalization, infection). Some subjects cannot wait too long for treatment and evaluation, since a long wait may result in occurrence of the clinical outcome. Scheduling the order of subjects to allocate to the required limited resources is a challenging technical problem.

One exemplary technical problem that arises is from the perspective of the main medical intervention (e.g., primary care physician) which is responsible for managing a group of subjects (e.g., patients). Different subjects may have different medical conditions, may be on different specific priority lists for treatment and/or evaluation by different medical resources, and/or be at different risks for developing clinical outcomes. Reduction and/or prevention of clinical outcomes through treatment and/or evaluation by different medical resources is being sought. The challenge is how to prioritize the treatment and/or evaluation by the main medical intervention in view of the state of the subjects on the various specific priority lists, for example, to provide holistic and preventive medical care for the subjects where the most urgent cases are prioritized overall and/or where subjects are most likely to improve from the intervention. This is challenging, since it may not be clear which subjects are the most urgent overall cases and/or which subjects are most likely to benefit, from the different specific priority lists. Since the main medical resource is limited (e.g., the family physician has a maximum amount of time, and is limited in the number of patients that may be seen on any one day, and/or whose clinical file may be reviewed without necessarily requiring presence of the subject (e.g., clinical file is reviewed without a face to face (f2f) treatment and/or evaluation session, such as off-line)), the prioritization and/or ordering of subjects may be relative rather than absolute, which poses a technical challenge—how to objectively order the subjects for treatment and/or evaluation.

The problem of providing a uniform way to prioritize subjects is especially difficult in subjects that with different health needs that require multiple medical interventions, for example, subjects with multiple comorbidities and/or subjects with complex diseases. For example, a subject with diabetes may also suffer from heart disease, kidney disease, and COPD. Such subject may require a cardiac catheterization, cancer screening for lung disease, and management by a kidney specialist, in order to reduce risk of a clinical outcome.

At least some implementations of the systems, methods, apparatus, and/or code instructions described herein generated a combined priority list from multiple specific priority lists and/or compute a maximal follow up time interval for follow up of how the subject is being medically managed, to provide a holistic management of the subjects. The holistic management may reduce likelihood of clinical outcomes occurring to the subjects, by making sure that the subjects are followed up within the maximal time interval, and/or that subjects are prioritized according to the combined priority list. For example, the general practitioner (e.g., family physician, internist) managing the subject may review the subject's EMR according to the combined priority list to determine whether the subject is being medically managed as needed.

In at least some implementations, clinical files of subjects on one or more specific priority lists are managed (e.g., by a specific clinical intervention and/or by the main clinical intervention) without necessarily requiring presence of the subject (e.g., clinical file is reviewed without a face to face (f2f) treatment and/or evaluation session, such as off-line). For example, an oncologist may review clinical files of patients on a specific priority list of subjects that have not completed work-up for cancer, without requiring that the patient be present.

At least some implementations of the systems, methods, apparatus, and/or code instructions described herein improve the technical field of automated processes to assist in allocation of subjects to limited medical resources, for example, providing a GUI to assist in the allocation.

At least some implementations of the systems, methods, apparatus, and/or code instructions described herein address the above mentioned technical problem(s) by training multiple ML models using respective specific priority lists, to each generate a respective outcome of a respective target priority list of a sub-set of subjects for prioritized evaluation and/or treatment for a respective target medical outcome (e.g., by a respective specific medical intervention) and/or for treatment and/or evaluation by the respective specific medical intervention (e.g. for the respective target medical outcome) in response to an input of data extracted from EMR of at least the sub-set of target subject, and by providing a combined prioritization component for generating an outcome of a target combined priority list of target subjects for prioritized evaluation and/or treatment in response to an input of outcomes of the plurality of specific machine learning models. The combined prioritization component may be another trained ML model trained on a main training dataset and/or aggregation code that computes an aggregated score for each subject on multiple specific lists, and creates the combined list by ranking the subjects according to aggregated scores.

At least some implementations of the systems, methods, apparatus, and/or code instructions described herein provide an optimal allocation of subjects to limited medical resources, by taking account of the current risk state of the subjects, and optimizing the likelihood of the subjects being evaluated and/or treated before a clinical outcome occurs. At least some implementations of the systems, methods, apparatus, and/or code instructions described herein use the outcomes of the ML models and/or the combined prioritization component, for generating a target combined priority list of target subjects for treatment and/or evaluation (e.g., by a main medical intervention) by combining multiple specific priority lists of different sub-sets of the target subjects for treatment and/or evaluation by different specific medical interventions, where the order of the target subjects on the target combined priority list represents the most optimal order for treatment and/or evaluation by the main medical intervention for reducing risk of clinical outcome in the subjects. By following the order of subjects on the target combined priority list, the main medical intervention may provide the most effective treatment and/or evaluation early to the most at risk subjects, which may improve overall clinical outcomes for the whole set of subjects.

At least some implementations of the systems, methods, apparatus, and/or code instructions described herein improve the technology of ML architectures for dynamic prioritization of subjects for medical evaluation and/or treatment, for example, by a main medical intervention that is resource limited. The improvement is over, for example, treating each subject individually by computing an absolute score for each subject directly from the raw data (e.g., extracted from EMRs) and then treating subjects according to the absolute score. In at least some implementations, the improvement provided is by first computing (and/or accessing) multiple respective specific priority lists computed by multiple ML models from data extracted from EMRs. Each respective specific priority list is for a different sub-set of subjects and for treatment and/or evaluation by different medical interventions. Now, the multiple specific priority lists are combined by the combined prioritization component into a combined priority list, which is for treatment and/or evaluation of the set of subjects by another medical intervention (e.g., the main medical intervention). The combined prioritization component may be implemented as another trained ML model that generates the combined priority list as an outcome in response to an input of multiple specific priority lists. The ML model may generate the combined priority list as a single simultaneous outcome for the set subjects, rather than treating each subject independently. Alternatively or additionally, the combined prioritization component may be implemented as aggregation code that computes a weighted aggregated numerical score for each subject on the multiple specific priority lists, and ranks the subjects according to respective weighted aggregated numerical score to generate the combined priority list.

At least some implementations of the systems, methods, apparatus, and/or code instructions described herein improve over standard approaches, for example, manual approaches, and/or approaches that prioritize and/or rank subjects based on medical information. Manual approaches do not result in the most optimal solution (i.e., allocation of subjects to limited resources before a clinical outcome occurs) since a human cannot consider all available information in the decision making process. Such manual processes, which are prone to bias and/or take time, are generally based on a reactive approach of “putting out fires”, i.e., prioritizing emergencies, rather than taking a proactive approach and reviewing the EMR of subjects prior to clinical outcomes occurring in order to reduce likelihood of the clinical outcomes. Other automated approaches that simply prioritize and/or rank subjects based on medical information fail to account for subjects with multiple morbidities that require a global management for multiple treatments and/or evaluations. Such subjects with multiple morbidities require a combined priority list that takes account of how each subject is ranked within multiple specific priority lists, which is cannot be performed by a simple single list.

At least some implementations of the systems, methods, apparatus, and/or code instructions described herein may improve health outcomes in subjects (e.g., reduce clinical outcomes, increase quality of life, increase lifespan), improve medical experience, and/or reduce overall healthcare costs, by generating the combined priority lists of subjects, and/or by generating the maximal time interval for follow up of subjects. The combined priority list may be used to provide overall holistic care of the subject by a certain healthcare provider (e.g., doctor, nurse, social worker, dietician, pharmacist, physiotherapist), for example, in order to prioritize those subjects most likely to benefit from medical treatments and/or intervention, and/or those subjects that most need the medical treatments and/or interventions.

At least some implementations of the systems, methods, apparatus, and/or code instructions described herein relate to a unified, interactive, and/or user-friendly platform for proactive medicine. The information described herein (e.g., specific and/or combined priority lists) may be dynamically updated, for example, daily. The end user (e.g., sometimes referred to herein as main medical intervention) may be, for example, primary care physicians and/or nurses, but may include additional healthcare providers (e.g., pediatricians, social workers). Providers (e.g., main medical interventions) may use embodiments described herein to tackle proactive healthcare needs in an organized and efficient way. Risk ranking of the patients, both within each specific priority list and in the combined list, may allow providers to focus their efforts on the most vulnerable population. The information in the medical records may be used in combination with up-to-date clinical guidelines to yield individualized recommendation for action for individuals included in the priority lists. Connecting to electronic health records, may allow providers to act on the recommendations (e.g., refer the patient to the recommended lab work) with one click, saving up work time and reducing the administrative burden. The combined list may be used to yield a maximal clinical benefit under severe time constraint. A provider with little time for proactive work may focus their efforts on the individuals marked as highest risk in the combined priority list. Providers may customize the weight assigned to each specific list within the combined list, thus allowing fitting the combined list to local health needs. A specific sub-set of subjects on a specific list can be designated as “in focus”, i.e. those deemed as higher priority for intervention. Provider autonomy may be retained. Physicians may remove patients from lists (based on pre-set criteria for exclusion), as well as set individual follow-up time to return patients to the list. The automated follow-up frequency (e.g., maximal recommended time interval) for healthcare providers (e.g., nurses) may be set up providing the rules defining the interval after which a patient will be returned to the list for additional assessment. Nurses may not set up a follow-up date that does not comply with these rules, i.e., later than the maximal recommended time interval. It is noted the automated follow-up frequency is based on risk parameters, which are not necessarily obtained from a specific priority list. For example, the risk parameters may be computed by another process, for example, a set of rules and/or another machine learning model.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

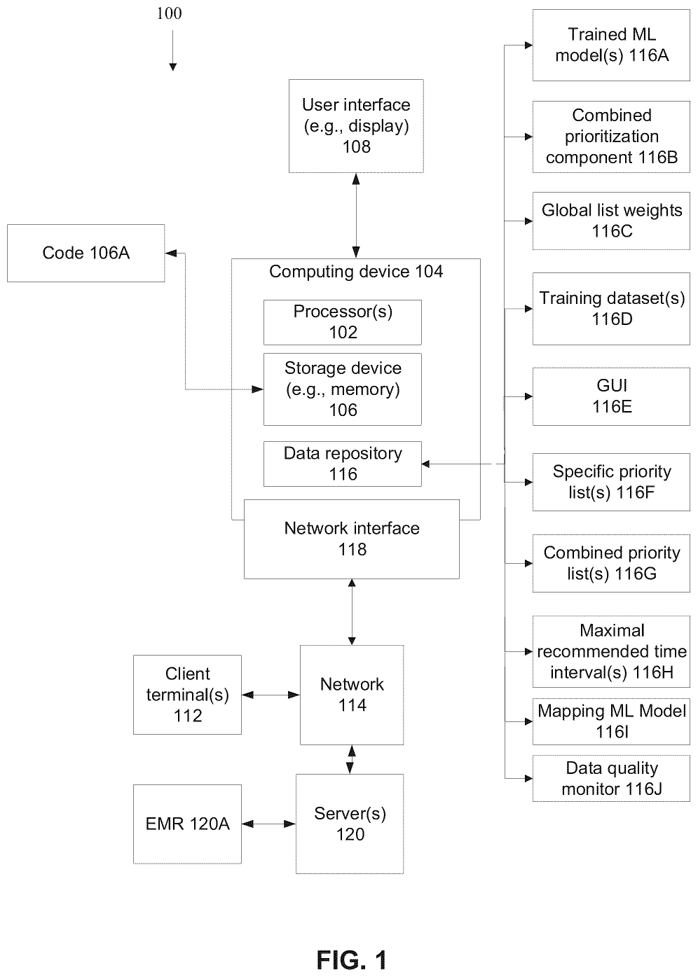

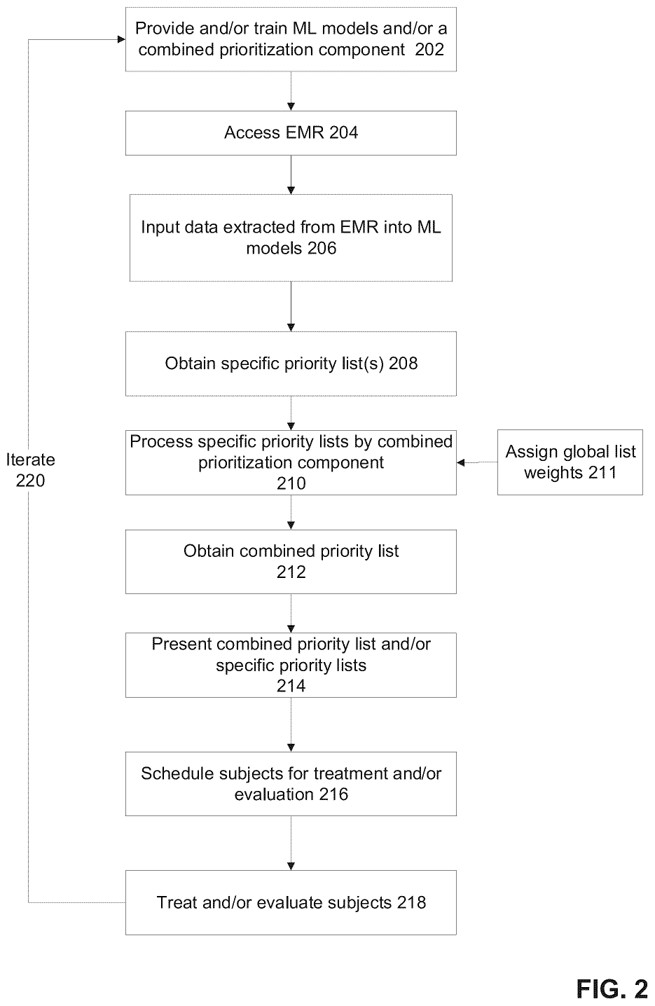

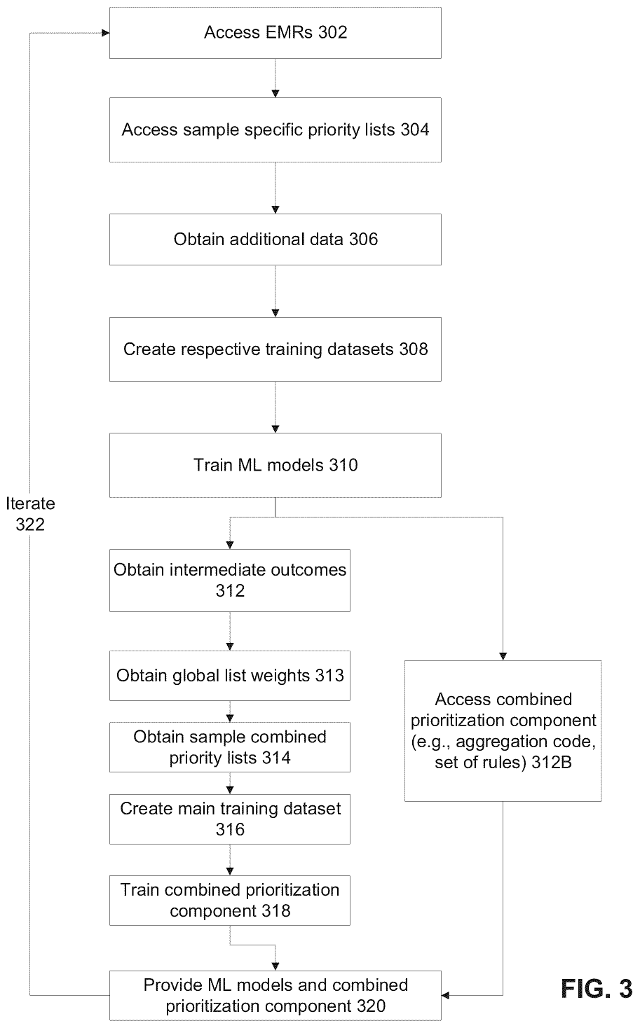

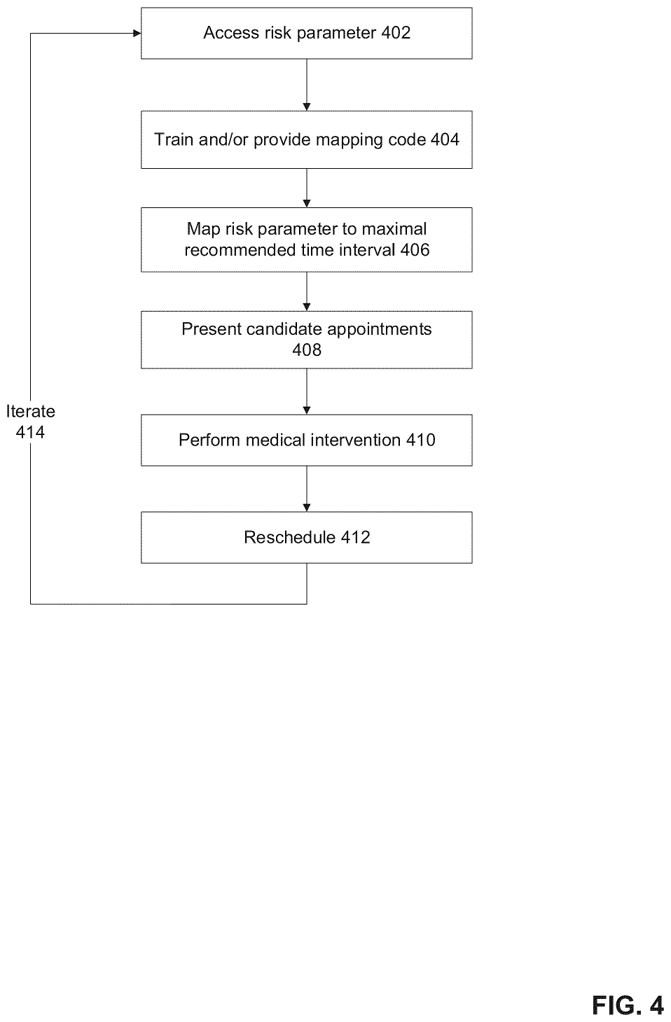

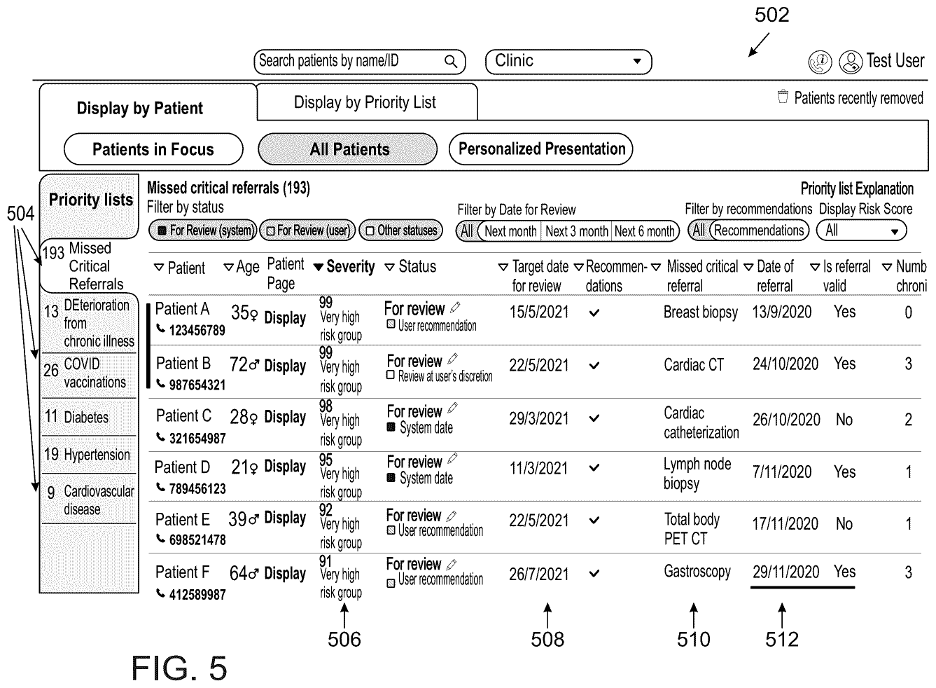

Reference is now made to FIG. 1, which is a block diagram of a system 100 for computing a combined priority list 116G from multiple specific priority lists 116F each computed from data extracted from EMRs 120A (and/or other sources of individualized medical information, for example, imaging systems, laboratory systems, pharmacy purchases), and/or computing maximal recommended time intervals 116H for performing a respective medical intervention, in accordance with some embodiments of the present invention. Reference is also made to FIG. 2, which is a flowchart of a method of prioritization of subjects for treatment and/or evaluation (e.g., by the main medical intervention that is resource limited), in accordance with some embodiments of the present invention. Reference is also made to FIG. 3, which is a flowchart of a method of training multiple machine learning models and/or a combined prioritization component for dynamic prioritization of subjects for evaluation and/or treatment (e.g., by a main medical intervention that is resource limited), in accordance with some embodiments of the present invention. Reference is also made to FIG. 4, which is a flowchart of a method of computing maximal recommended time intervals for evaluation and/or treatment of subjects (e.g., by a main medical intervention that is resource limited), in accordance with some embodiments of the present invention. Reference is also made to FIGS. 5-10, which are screen shots of an exemplary GUI, in accordance with some embodiments of the present invention.

System 100 may implement the acts of the method described with reference to FIGS. 2-10, by processor(s) 102 of a computing device 104 executing code instructions 106A stored in a storage device 106 (also referred to as a memory and/or program store).

Multiple architectures of system 100 based on computing device 104 may be implemented. In an exemplary implementation, computing device 104 storing code 106A may be implemented as one or more servers (e.g., network server, web server, a computing cloud, a virtual server) that provides centralized services for computing specific priority lists 116F, combined priority lists 116G, and/or maximal recommended time intervals 116H, optionally from data extracted from EMR 120A of subjects. For example, respective client terminals 112 may access computing device 104, optionally GUI 116E (e.g., web site, GUI code), over a network (e.g., public network, private networks, intra-nets, virtual private networks, the internet, cellular networks, wireless networks, LANs, and the like). The computed specific priority lists 116F, combined priority lists 116G, and/or maximal recommended time intervals 116H may be presented on respective displays of client terminals 112, for example, within a GUI (e.g., running GUI code 116E). In another example, computing device 104 provides software as a service (SaaS) to the client terminal(s) 112, provides software services accessible using a software interface (e.g., application programming interface (API), software development kit (SDK)), provides an application for local download to the client terminal(s) 112, and/or provides functions using a remote access session to the client terminals 112, such as through a web browser. In another example, computing device 104 may include locally stored software (e.g., code 106A) that performs one or more of the acts described with reference to FIG. 2-10, for example, as a self-contained client terminal. In such implementation, the specific priority lists 116F, combined priority lists 116G, and/or maximal recommended time intervals 116H may be locally computed. The ML models and/or combined prioritization component described herein may be locally trained, and/or centrally trained and obtained from the centrally training device. In yet another implementation, code 106A may be an add-on to an existing electronic medical record (EMR) management application that manages EMR 120A, for example, run and/or stored by a server 120 (e.g., EMR server, healthcare provider server). In yet another implementation, code 106A may be integrated with GUI 116E into an application that may be remotely and/or locally accessed. Code 106A may be remotely accessed via an interface such as an API and/or SDK.

Computing device 104 may be implemented as, for example, a client terminal, a server, a computing cloud, an EMR server, a virtual server, a virtual machine, a mobile device, a desktop computer, a thin client, a Smartphone, a Tablet computer, a laptop computer, a wearable computer, glasses computer, and a watch computer.

Processor(s) 102 of computing device 104 may be implemented, for example, as a central processing unit(s) (CPU), a graphics processing unit(s) (GPU), field programmable gate array(s) (FPGA), digital signal processor(s) (DSP), and application specific integrated circuit(s) (ASIC).

Processor(s) 102 may include multiple processors (homogenous or heterogeneous) arranged for parallel processing, as clusters and/or as one or more multi core processing devices. Processor(s) 102 may be arranged as a distributed processing architecture, for example, in a computing cloud, and/or using multiple computing devices. Processor(s) 102 may include a single processor, where optionally, the single processor may be virtualized into multiple virtual processors for parallel processing.

Data storage device 106 stores code instructions executable by processor(s) 102, for example, a random access memory (RAM), read-only memory (ROM), and/or a storage device, for example, non-volatile memory, magnetic media, semiconductor memory devices, hard drive, removable storage, and optical media (e.g., DVD, CD-ROM). Storage device 106 stores code 106A that implements one or more features and/or acts of the method described with reference to FIGS. 2-10 when executed by processor(s) 102.

Computing device 104 may include a data repository 116 for storing data, for example, storing one or more of: trained ML models 116A that generate the outcome of respective specific priority lists, combined prioritization component 116B that generate the outcome of the combined priority list, set of rules 116B, global list weights 116C which may be assigned to different specific priority lists for combination into the combined priority list (it is noted that there may be multiple sets of global list weights 116C, such as per user, since different users may select different customized sets of global list weights), training dataset(s) 116D documenting data for training ML models 116A and/or combined prioritization component 116B, GUI 116E, specific priority list(s) 116F, combined priority list(s) 116G, and/or maximal recommend time interval(s) 116H, mapping ML model 1161, and/or a data quality monitor 116J component for monitoring of EMR data quality and stability, and degradation of ML model performance, as described herein. Data repository 116 may be implemented as, for example, a memory, a local hard-drive, virtual storage, a removable storage unit, an optical disk, a storage device, and/or as a remote server and/or computing cloud (e.g., accessed using a network connection).

Computing device 104 may include a network interface 118 for connecting to network 114, for example, one or more of, a network interface card, a wireless interface to connect to a wireless network, a physical interface for connecting to a cable for network connectivity, a virtual interface implemented in software, network communication software providing higher layers of network connectivity, and/or other implementations.

Network 114 may be implemented as, for example, the internet, a local area network, a virtual private network, a wireless network, a cellular network, a local bus, a point to point link (e.g., wired), and/or combinations of the aforementioned.

Computing device 104 may connect using network 114 (or another communication channel, such as through a direct link (e.g., cable, wireless) and/or indirect link (e.g., via an intermediary computing unit such as a server, and/or via a storage device) with client terminal(s) 112 and/or server(s) 120 and/or other computing devices, as described herein.

Computing device may access EMR 120A for extracting data for inputting into ML models 116A for computing the specific priority lists, for example, via server 120 and/or via network 114.

Computing device 104 and/or client terminal(s) 112 include and/or are in communication with one or more physical user interfaces 108 that include a mechanism for a user to enter data (e.g., define weights) and/or view the computed lists 116F-G and/or maximal recommend time intervals 116H. Exemplary user interfaces 108 include, for example, one or more of, a touchscreen, a display, a keyboard, a mouse, and voice activated software using speakers and microphone. GUI 116E may be presented on user interface 108.

As described herein, lists 116F-G may be according to target clinical outcomes, and/or according to specific medical interventions. Lists according to target clinical outcomes may be for treatment and/or evaluation by one or more specific medical interventions and/or by other interventions. Lists according to specific medical interventions may be for treatment and/or evaluation of target clinical outcomes and/or other reasons.

Lists 116F-G may include personalized recommendations. For example, when lists are for target clinical outcomes, the personalized recommendations may be for treatment and/or evaluation by one or more specific medical interventions. A specific recommendation may appear in one or more priority lists.

ML models 116A may be provided for generation of specific priority lists and/or for the combined priority list, as described herein. It is noted that one or more set-of-rules may be used in addition to trained ML model(s) 116A.

ML models 116A and/or ML mode implementation of combined prioritization component 116B described herein may be implemented as, for example, one or more classifiers, statistical classifiers and/or other statistical models, neural networks of various architectures (e.g., fully connected, deep, encoder-decoder, recurrent), support vector machines (SVM), logistic regression, k-nearest neighbor, decision trees, boosting, random forest, a regressor, and/or any other commercial or open source package allowing regression, classification, dimensional reduction, supervised, unsupervised, semi-supervised or reinforcement learning. Machine learning models may be trained using supervised approaches and/or unsupervised approaches.

One or more ML models 116A described here may be an aggregation of multiple ML models, a combination of multiple ML models, and/or an ensemble of multiple ML models.

ML models may be descriptive, inference, predictive and/or causal.

Optionally, the specific ML models and/or the combined prioritization components include one or more architectures designed to process sequential data such as lists and/or time sequences, for example, at least a recurrent neural network (RNN) component, graph neural network (GNN) component, and/or other architectures such as embedding approaches. The RNN may be trained to obtain an input of, and/or generate an input of, an ordered list of subjects which may be time correlated, as described herein.

Referring now back to FIG. 2, at 202, multiple machine learning models and/or combined prioritization component are trained and/or provided. An exemplary process for training the machine learning models and/or combined prioritization component is described with reference to FIG. 3.

Alternatively or additionally, other code for generating specific and/or priority lists is provided and/or defined, for example, sets of rules, and/or weights

At 204, a set of target EMRs of target subjects is accessed. Data is obtained (e.g., extracted from the EMRs). The data obtained from the EMRs may be according to expected input into the ML models and/or data analyzed by sets of rules.

It is noted that EMRs are used as an exemplary implementation that is not necessarily limiting, since other data from other sources may be obtained, for example, other databases and/or manually entered.

Exemplary data includes: blood test results, imaging results, other laboratory test results, current medications, previous medications, current diagnoses, previous diagnoses, previous medical procedures (e.g., surgery), previous hospitalizations, demographic profile (e.g., age, biological gender), previous clinic appointments, and/or clinical markers such as height, weight, BMI.

The target subjects may be, for example, patients of a primary care giver, such as family physician.

Optionally, a derivation dataset is computed from data extracted from the EMRs. The derivation dataset may include multiple sets of combinations of processed and/or unprocessed (i.e., raw) features. The raw features are obtained by data directed extracted from the EMRs, for example, Hemoglobin level of 14.3, and 23 pack-year history of smoking. Processed features are extracted by applying feature functions to data extracted from the EMR. Data may be values obtained at different times for the same field of EMR, for example, average Hemoglobin over the last 5 years, and maximal and minimal fasting blood glucose level over the last 2 years. Other feature functions may be combinations of two or more fields of the EMR, for example, an indication of whether the subject is a smoker and has high LDL (low density lipoprotein) (e.g., binary feature indicating “yes” or “no”). Features may be, for example, a numerical value, a binary value, and/or a classification category. Processed features may be computed from two or more unprocessed features.

At 206, the data obtained from the set of target EMRs (e.g., the derivation dataset) is fed into the specific ML models. Different subsets of the data may be fed into different specific ML models, according to the expected input into each respective specific ML model.

Data of different sub-sets of target subjects may be fed into different specific ML models. For example, for subjects not diagnosed with COPD, no data is fed into a certain specific ML model that predicts severe exacerbation of COPD. Alternatively, data of all target subjects is fed into all specific ML models.

Alternatively or additionally, one or more specific priority lists are computed by applying a set of rules to the respective sub-set of data extracted from the EMRs of each respective sub-set of subjects. The set of rules may be predefined, and/or adjustable by the user, for example, using a GUI.

It is noted that using a set of rules is an exemplary not necessarily limiting implementation, and other computational code may be used.

At 208, multiple specific priority lists are obtained as outcomes of the specific ML models and/or computed by the set of rules.

Optionally, each respective specific ML model generates the respective specific priority list as a respective single simultaneous outcome, i.e., the respective specific priority list is outputted as whole ranked list by the respective specific ML model. A ranking within the respective specific priority list of a respective subject may be relative to other subjects within the list.

Specific priority lists define an ordering and/or ranking of the respective subjects on the list, implemented, for example, as a linked list, an ordered list, and/or a ranking based on scores associated with each subject on the list. The ordering may be relative, where each subject is ordered relative to other subjects. Alternatively or additionally, ordering is based on absolute scores, such as risk scores, where there may be multiple subjects with the same scores that are at the same ranking in the order of the list.

Optionally, each respective specific priority list includes a respective sub-set of the target subjects (or all of the target subjects) scheduled in a prioritized sequence for treatment and/or evaluation by a respective specific medical intervention. Each respective medical intervention is resource limited (e.g., in terms of time and/or availability, such as number of subjects that may be treated and/or evaluated per unit time, within amount of time available for the treatment and/or evaluation), in that the number of available resources is much less than the number of target subjects that require the respective medical intervention. A small subset of the target subjects may access the respective medical intervention at any one time, and therefore target subjects are prioritized for sequential treatment and/or evaluation.

Exemplary specific priority lists may be generated for specific medical interventions. The specific medical interventions may be for treatment and/or evaluation of target clinical outcomes, as described herein. Examples of specific medical interventions include: surgical procedure (e.g., bariatric surgery), catheterization procedure (e.g., stent placement, diagnostic procedure), laboratory testing (e.g., genetic sequencing, biopsy), lifestyle modification coaches (e.g., dietician, smoking cessation support groups), medical imaging (CXR, MRI, CT), non-imaging work-up (e.g. laboratory work-up), cancer screening test (e.g., colonoscopy, mammography, low dose CT chest scan for pulmonary nodules), immunizations (e.g. COVID vaccine for high-risk individuals) and medical specialist consultation (e.g., orthopedic surgeon, cardiologist, endocrinologist.

Alternatively or additionally, specific priority list(s) are for target clinical outcomes. Subjects on the specific priority list(s) may be treated and/or evaluated for the target clinical outcome(s) by the specific medical intervention(s) described herein. Examples of clinical outcomes include: heart attack, stroke, hospitalization, fall, COPD exacerbation, severe COVID, osteoporotic fracture, diabetes complications. Each target subject in each of the specific priority lists may be associated with a respective numerical score indicative of risk of the respective target clinical outcome (e.g., diagnosis of a respective clinical condition). For example, subjects with higher numerical scores have a higher risk of the target clinical outcome than subjects with lower numerical scores. Alternatively or additionally, the specific priority lists and/or the combined priority list are each arranged as a respective ordered ranking of the respective sub-set of the target subjects. A certain ranking within a respective list is relative to other subjects within the respective list. For example, subjects higher up on the list have a higher risk of a target clinical outcome than subjects lower down on the list. The ranking of subjects may be according to numerical scores, where subjects with higher scores are placed higher on the list than subjects with lower scores. Examples of diagnoses of clinical conditions include: cancer (e.g., lung, colon, prostate, breast), cardiovascular disease, osteoporosis, and type 2 diabetes.

Examples of specific priority lists include: subjects high risk for severe COVID-19 who have neither been vaccinated nor have been infected, individuals at risk for deterioration of an underlying medical condition, subjects at risk for osteoporotic fractures, subjects with diabetes, and subjects with cardiovascular disease.

Other examples of specific priority lists include: subjects who were referred to additional work-up that was included as priority work-up (e.g., work up for suspected malignancy, work up after a cerebral infarction, etc.), and did not complete the work up within a specified time frame (e.g., 3-6 months from the referral date), and/or subjects taking specific medications necessitating a specific follow-up (e.g. blood work).

Specific priority lists may be associated with one or more recommendations. For example, to perform blood work for the subjects taking medications necessitating follow-up, to be vaccinated for subjects at high risk for severe COVID-19, and to perform a bone density scan and/or take bisphosphonates for subject at risk for osteoporosis.

The specific priority lists may be for treatment and/or evaluation using an offline (i.e., without presence of the subject) review of the subject's medical records, for example, to check whether the subject is compliant with medications and/or tests and if not try to figure out why, whether the subject is scheduled for required procedures and/or evaluations, and/or whether other changes to the subject's health plan are needed (e.g., change medications, undergo procedures and/or tests, lifestyle changes).

At 210, the multiple specific priority lists are processed by the combined prioritization component for example, implemented as a trained ML model and/or implemented as aggregation code (e.g., a set-of rules).

A combined priority list may be generated by combining multiple specific priority lists. The combination may be according to a set of rules. Optionally, a respective aggregated score is computed for each respective subject according to respective numerical scores of the respective subject on multiple specific priority lists. The combined priority list includes the subjects ranked by respective aggregated scores.

At 211, optionally, each of the priority lists is assigned a respective global list weight. The global list weight indicates a magnitude of prioritization of the respective target specific priority list. The magnitude may be absolute, for example, a value of 4, or 8, or 1.3, or other values. The magnitude may be relative, for example, a first priority list is assigned a global list weight that is 2.25× the global list weight of a second priority list.

The global list weights may be represented as numerical values, which may be discrete (e.g., 2, 4, 8), and/or continuous optionally within a predefined number of decimal places, for example, a single decimal (e.g., 1.2, 5.5), two decimal places (e.g., 2.25, 4.37), three decimal places (e.g., 8.745, 2.324), or more. The global list weights may be represented as classification categories, for example, low magnitude, medium magnitude, and high magnitude.

The global list weights may be selected according to a geographical location. For example, in one neighborhood where covid infection rates are high, the global list weights for clinics located that that neighborhood are high for the covid infection specific priority list. In another neighborhood where covid infection rates are low, the global list weights for clinics located that that neighborhood are low for the covid infection specific priority list.

The global list weights may be defined according to a set of rules. For example, the global list weight for a specific priority list of heart attack is one or more of: computed according to a certain equation, cannot be more than 2.5× the global list weight defined for a specific priority list of diabetes, and/or is limited by the range 2.5-4.5, and the like.

The global list weights may be fixed within a defined range of values, for example, manually set by an administrator, and/or automatically computed by code such as based on a mathematical function. The defined range may prevent users from setting weights too high or too low, to prevent situations in which patients at high priority of certain medical conditions are listed low on the combined priority list. For example, the global list weight for CT scans is within the range of 1-2.5, and the global list weight for cardiac catheterization is 2.5-5, to prevent those that need CT scans from being higher on the combined list than those needed cardiac catheterization.

The global list weights may be constrained by one or more imposed criteria. Criteria may restrict the selection of global list weights, for example, within a high threshold, a low threshold, a set of rules, and/or location-based. Different criteria may be imposed on different global list weights, for example, a respective criterion pre global list weight. The same criteria may be imposed on global list weights of the same specific priority list for different users. For example, the global list weight of a specific priority list indicating risk of CVD is restricted to 1.0-1.5 for all users. Alternatively or additionally, the imposed criteria may be per user, even for the same specific priority list. For example, for a cardiologist the global list weight of the specific priority list indicating risk of CVD is restricted to 1.6-1.9, while for a rheumatologist the global list weight of the specific priority list indicating risk of CVD is restricted to 0.1-0.3. Alternatively or additionally, the imposed criteria may be location-based. For example, for a general practitioner located in a dense downtown urban center, the global list weight of the specific priority list indicating risk of flu is restricted to 2-5, while for another general practitioner located in a rural setting, the global list weight of the specific priority list indicating risk of flu is restricted to 0.5-0.9.

The global list weights may be adjustable to different values by different users, for example, using a user interface such as a GUI. The global list weight may be dynamically adjusted. The global list weights may be set in advance, for example, by the user and/or administrator, and saved for future sessions.

The respective aggregated score is computed by multiplying, for each respective subject, each respective numerical score of each respective priority list by the respective global list weight assigned to the respective list to compute a respective weighted score for the respective list. The same global list weight is applied to all scores of all subjects of the same specific priority list. The aggregated score is computed by summing the weighed scores of each specific priority list for each person. The weights assigned to the different specific lists may add to a total of 1 (e.g., indicating relative weights) and/or the weights assigned to the different specific lists may be independent absolute weights (e.g., do not add to a specific target value such as 1). In an example, list 1 indicates risk for severe COPD, list 2 indicates likelihood of deterioration of chronic disease, and list 3 indicates subjects that did not complete crucial work-up (e.g., for evaluation of cancer and/or heart disease). For example, a person with a score of 0.8 on list 1, 0.4 on list 2, and 0.9 on list 3, where list 1 has a weight of 0.6, list 2 has a weight of 0.3, and list 3 has as weight of 0.1 (e.g., relative weights), has an aggregated score of 0.8*0.6+0.4*0.3+0.9*.01=0.69. In another example (e.g. absolute weights), list 1 has a weight of 10, list 2 has a weight of 10 and list 3 has a weight of 50.

Each respective weight may be dynamically adjustable by a user, for example, via a GUI presented on a display. Alternatively or additionally, each respective weight may be preset, for example, by the user during a previous session, by another user, by an administration, and/or automatically computed by code.

Alternatively or additionally the specific priority lists are fed into the trained ML model implementation of the combined prioritization component. The specific priority lists may be fed as whole lists into the trained ML model implementation of the combined prioritization component. For example, a two dimensional dataset that preserves the rankings of subjects is created, where each row indicates a respective specific priority list, and each column indicates a respective rank of the respective subject within the respective specific priority list. A numerical score may be assigned. The two dimensional dataset may be fed into the combined prioritization component.

Optionally, one or more specific priority lists are weighted by respective global list of weights. Numerical values of subjects in the respective specific priority list may be weighted by the respective global list weight. The weights may be assigned to each priority list by a combination component of the ML model ensemble.

At 212, a combined priority list is obtained as an outcome of the combined prioritization component.

The combined priority list indicates a prioritized ranking of subjects ranked for evaluation and/or treatment (e.g., by a main medical intervention, for example, a medical provider consultation, for example, of a pediatrician, a primary care giver, an internist, a geriatrician, and/or a family physician, which has limited time availability). The medical provider consultation may be an offline (i.e., without presence of the subject) review of the subject's medical records, for example, to check whether the subject is compliant with medications and/or tests, whether the subject is scheduled for required procedures and/or evaluations, and/or whether other changes to the subject's health plan are needed (e.g., change medications, undergo procedures and/or tests, lifestyle changes).

Optionally, the combined prioritization component generates the combined priority list as a respective single simultaneous outcome, i.e., the combined list is outputted as whole ranked list by the combined prioritization component. A ranking within the combined list of a respective subject may be relative to other subjects within the list.

Alternatively, the combined prioritization component generates a respective numerical score for each respective subject. The combined priority list is created by ranking subjects according to respective numerical scores.

At 214, a GUI presenting the specific priority lists and/or the combined priority list is generated and/or presented on a display.

Optionally, a user may manually adjust the global list weights and/or ranking and/or scores of subjects in the specific priority lists and/or the combined priority list via the GUI. The changes to the weights and/or in ranking to the specific priority lists and/or the combined priority list made by the user may be monitored. In response to a detected change, the respective specific training dataset and/or the main training dataset used to train the specific ML model and/or the combined prioritization component may be updated. Training of the specific ML models and/or the combined prioritization component may be updated using the updated dataset. The dynamic updating of the training datasets and/or dynamic training “learns” preferences of the users, and/or “learns” mistakes in computing the rankings, for computation of future lists.

At 216, optionally subjects are scheduled for treatment and/or evaluation by respective specific medical interventions according to respective specific priority lists, and/or subjects are prioritized and/or scheduled for evaluation and/or treatment (e.g., by the main medical intervention).

The scheduling may be prioritized according to respective ranking and/or respective scores of each subject within each respective list.

Optionally, the target specific priority lists and/or the target combined priority list are time correlated priority lists. Each time correlated priority list indicates for each respective ranked subject, a maximal recommended time interval for performing a corresponding medical intervention for reducing or preventing a target clinical outcome. The maximal time interval may be a value (e.g., numerical, date), outputted by the specific and/or combined prioritization components, and stored in association with the respective rank. For example, no more than 24 hours, or 3 days, or 7 days, or 2 weeks, or 1 month, or 3 months, or 6 months, or 1 year. Alternatively or additionally, the maximal time interval may be defined by the respective rank within the respective list. For example, subjects in the first rank require immediate attention (e.g., today), subjects in the second rank can wait a week, subjects in the third rank can wait a month, etc.

Scheduling may be performed automatically, for example, appointments are automatically created and stored in electronic calendars of the subjects and/or of the respective medical interventions, for example, stored in a health management system used by subjects and healthcare providers.

Optionally, user may change the automatically scheduled appointment, and/or book their own appointment. Optionally, users cannot schedule and/or change appointments to dates later than the maximal time interval. The maximal time interval may indicate a repeated frequency for performing the evaluation and/or treatment. For example, a nurse which has diabetic patients that undergo repeated blood tests to monitor the diabetes cannot schedule review appointments to review the blood test results greater than the maximal time interval frequency (e.g., at least once a month).

Optionally, scheduling may be performed according to scheduling data indicating availability of the main medical intervention, for example, daily appointment timeslots for different dates. Optionally, the target specific priority lists and/or the target combined priority list are time correlated priority lists. The priority lists may be generated in response to an input of the scheduling data (e.g., fed into the respective ML models and/or into the combined prioritization component which may be trained on respective training datasets that includes sample scheduling data). Such time correlated priority lists may include a candidate appointment for the respective medical intervention according to the scheduling data, for example, appointments may be automatically generated according to the capacity of the respective intervention on different dates. Alternatively or additionally, each respective rank within each respective time correlated priority list may indicate a recommended maximal time interval for performing a corresponding medical intervention on the respective subject. The recommended maximal time interval may be computed, for example, as described with reference to FIG. 4. The recommended maximal time interval may be for an effective allocation of resources for performing the respective medical intervention based on an input of a schedule of availability of resources for performing each respective corresponding medical intervention for each of multiple time intervals.

At 218, the target subjects are sequentially treated and/or evaluated by the respective specific medical interventions and/or by the main medical inventions according to the prioritized order defined by the specific priority lists and/or defined by the combined priority list, optionally, before each respective maximal time interval (e.g., frequency).

At 220, one or more features described with reference to 202-218 may be iterated. The iterations may be performed, for example, each time a user accesses the GUI that presents the computed lists, for re-computing and presenting the lists using updated data.

Iterations may be automatic and/or manual.

Iterations may be triggered, for example, by one or more of: date, new data, performance degradation, infrastructure changes, and/or user requests.

Optionally, the data of the EMRs that is extracted (e.g., as described with reference to 204) may be monitored to detect a change in at least one value of at least one field, for example, new test results, new diagnoses, change in medications, and the like. The iterations may be performed when the change is detected. For example, by feeding the updated data obtained from the EMR into the specific ML models for obtaining an updated set of specific priority lists, and feeding the updated specific priority lists into the combined prioritization component for obtaining an updated combined priority list.

Referring now back to FIG. 3, at 302, a set of target EMRs of target subjects is accessed. Data is obtained (e.g., extracted from the EMRs). The data obtained from the EMRs may be according to expected input into the ML models and/or data analyzed by sets of rules. Alternatively, for example, during initial training of the ML models when the relevance of data is not known, a much larger set of data may be extracted.

It is noted that EMRs are used as an exemplary implementation that is not necessarily limiting, since other data from other sources may be obtained, for example, other databases and/or manually entered.

Exemplary data includes: blood test results, imaging results, other laboratory test results, current medications, previous medications, current diagnoses, previous diagnoses, previous procedure (e.g., surgery), previous hospitalization, and demographic profile (e.g., age, biological gender).

The target subjects may be, for example, patients of a primary care giver, such as family physician.

Features may be extracted and processed from the EMRs, for example, as described with reference to 202 of FIG. 2.

At 304, for each specific medical intervention that is resource limited, a respective sample specific priority list of a respective sub-set of the subjects scheduled in a prioritized sequence for treatment and/or evaluation by the respective specific medical intervention, is accessed. Such sample lists represent a ground truth for training respective specific ML models.

The ground truth sample lists may be obtained, for example, by users manually ranking subjects (e.g., general practitioners, internists, pediatricians, for their patients), for example, via a GUI. In another example, the list is obtained as a schedule of for the respective specific medical intervention, for example, stored in a healthcare management system. For example, a list of future appointments of patients that are scheduled to undergo MRI scans.

Alternatively or additionally, one or more sample specific priority lists are represented as numerical scores assigned to each of the respective sub-set of subjects. Subjects may be ranked for creation of the list according to respective numerical scores, for example, subjects with relatively higher scores are placed higher on the list than subjects with relatively lower scores that are placed lower on the list. Scores may be manually determined and/or computed by code (e.g., set of rules, another trained ML model trained to compute scores). Scores may represent severity of the subject's condition, and/or risk probability of the subject experiencing the respective clinical outcome, or other values.

At 306, additional data used for ground truth labelling for training the specific ML models and/or for training the combined prioritization component may be obtained. For example, occurrence of a respective target clinical outcome, an indication of risk of a respective clinical outcome, a diagnosis of a respective clinical diagnosis, an indication of maximal recommended time interval by which the subject should be treated and/or evaluated by the respective intervention, and a history of previous time intervals between respective medical interventions.

Another example of additional data is a schedule of availability of resources for performing each respective corresponding medical intervention for each of multiple time intervals. For example, a schedule of when a certain PET/CT machine and related human resources to operate the machine are available to conduct imaging, such as Monday-Friday 9 AM-5 PM. Such scheduling data may be included in the training dataset for training the respective ML model for generating a respective time correlated priority list in response to an input of a schedule of time correlated availability of the respective resource for performing the respective medical intervention.

Another example of additional data is sample recommended maximal time intervals for treatment and/or evaluation of the respective subject by the respective specific medical resource, for example, no more than 24 hours, or 3 days, or 7 days, or 2 weeks, or 1 month, or 3 months, or 6 months, or 1 years. Additional exemplary details of recommended maximal time intervals may be provided with reference to FIG. 4.

The additional data may be automatically extracted from the EMR, for a healthcare management application used to view EMRs and/or schedule appointments for patients (and/or other datasets) and/or manually entered by a user, and/or computed by other code.

Optionally, the ML models are trained using the scheduling data to generate an outcome of time correlated priority lists (i.e., for the specific priority lists and/or for the combined priority list). Each respective rank within each respective time correlated priority list may denote a recommended maximal time interval for performing the corresponding medical intervention on the respective subject, for example, no more than 1 week, 2 weeks, 1 month, 3 months, or 6 months. The recommended maximal time interval may be an outcome indicating an effective allocation of resources for performing the medical intervention based on an input of a schedule of availability of resources for performing each respective corresponding medical intervention for each of multiple time intervals. The recommended maximal time interval may be generated, for example, as described with reference to FIG. 4.

At 308, a respective specific training dataset is created for each respective specific medical intervention and/or each respective risk of clinical outcome and/or each respective likelihood of diagnosis of future medical condition. Each respective specific training dataset that includes a respective subset of data extracted from the EMRs of at least a respective sub-set of subjects, and labeled with the specific priority list and/or labeled with other data such as scheduling data and/or sample maximal recommended time intervals.

At 310, each respective specific ML model is trained on its respective specific training dataset, for example, using supervised and/or unsupervised approaches.

Specific ML models are trained for generating an outcome of the respective target specific priority list of the sub-set of target subjects for prioritized evaluation and/or treatment by the respective specific medical intervention in response to an input of data extracted from the EMR of at least the sub-set of target subjects.

Specific ML model(s) may generate the respective target specific priority list as a whole, single, simultaneous outcome. Alternatively or additionally, specific ML model(s) may generate numerical scores for each subject (e.g., sequentially), and the respective list is generated (by the respective ML model, and/or by other code that receives the scores) by ranking subjects according to respective scores.

At 312, intermediate outcomes of intermediate specific priority lists are obtained from the trained specific ML models. For example, the trained specific ML models are fed data extracted from the EMRs (which may be different than the data used to train the specific ML models) to obtain the intermediate specific priority lists.

Each intermediate specific priority list may be for a certain sub-set of subjects.

Optionally, respective specific ML models may be for a respective clinical outcome and/or for a respective clinical diagnosis. Respective specific ML models may be trained to generate outcomes of respective target priority lists of the sub-set of target subjects at risk for the respective clinical outcome and/or diagnosed with the respective clinical diagnosis, for prioritized evaluation and/or treatment for the respective clinical outcome and/or respective clinical diagnosis by the respective specific medical intervention.

At 313, global list weights are obtained. Optionally, the intermediate specific priority lists are assigned respective global list weights, which are applied to numerical scores of each respective subject on the respective list. Additional details of the global list weights are described herein, for example, with reference to 211 of FIG. 2.

At 314, respective sample combined priority lists are obtained. Each sample combined priority list indicates subjects for prioritized evaluation and/or treatment (e.g., by the main medical intervention). Such sample combined priority lists represent a ground truth for training the combined prioritization component.

Each sample combined priority list may include the full set of subjects, for example, the union of the subset of subjects of the intermediate specific priority lists. For example, the full set of patients of the primary care physician, where different subsets of patients are scheduled for different treatments and/or evaluations.

The ground truth sample combined priority lists may be obtained, for example, by users manually ranking subjects (e.g., general practitioners, internists, pediatricians, for their patients), for example, via a GUI. In another example, the list is obtained as a schedule (e.g., for the respective main medical intervention), for example, stored in a healthcare management system. For example, a list of future appointments for review of medical files of patients by a primary care giver.

Alternatively or additionally, one or more sample combined priority lists are represented as numerical scores assigned to each of the respective sub-set of subjects. Subjects may be ranked for creation of the list according to respective numerical scores, for example, subjects with relatively higher scores are placed higher on the list than subjects with relatively lower scores that are placed lower on the list. Scores may be manually determined and/or computed by code (e.g., set of rules, another trained ML model trained to compute scores). Scores may be computed by aggregating, for each subject, their respective scores of the respective intermediate specific priority lists, optionally according to respective global list weights.

At 316, a main training dataset is created. The main training dataset includes the intermediate specific priority lists obtained as outcomes of the specific ML models, labeled with a respective combined priority list of the set of the subjects for treatment and/or evaluation (e.g., by the main medical intervention).

At 318, the combined prioritization component is trained using the main training dataset, for generating an outcome of a target combined priority list of target subjects for prioritized evaluation and/or treatment (e.g., by the main medical intervention) in response to an input of intermediate outcomes of the specific ML models. Each specific ML model is fed a subset of data extracted from the EMR of a respective subset of subjects, as described herein.

Alternatively to 312-318, at 312B, a combination prioritization component, optionally implemented as aggregation code such as a set of rules, is accessed. Such implementation represents a non-ML model implementation of the combination prioritization component.

At 320, multiple ML models and/or the combined prioritization component are provided. Optionally, an ML model ensemble is provided, which may include a selected combination of ML models and/or combined prioritization component.

Optionally, an intermediate component that assigns a respective preselected weight to each respective intermediate specific priority list to compute respective weighted specific priority lists is defined. The weighted specific priority lists are then fed into the combined prioritization component.

Different combinations of ML models and/or combined prioritization components may be provided, for example:
A full set including specific ML models, the combined prioritization component implemented as an ML model, and the intermediate component. For example, for computing the combined priority list using the ML models and allowing for adjustment of weights of the specific priority lists that are fed into the combined prioritization component implemented as an ML model.
The specific ML models and the combined prioritization component implemented as an ML model, excluding the intermediate component. For example, for computing the combined priority list using the specific ML models and the combined prioritization component implemented as an ML model.
The specific ML models, the intermediate component, and the combined prioritization component implemented as aggregation code. The combined priority list is computed using outcomes of the intermediate component that assign weights to the specific priority lists, as described herein.
The specific ML models excluding the intermediate component and including the combined prioritization component implemented as aggregation code.
The specific ML models excluding the intermediate component and excluding the combined prioritization component. For example, when the specific ML models are used to compute risk parameters which are mapped to maximal recommended time intervals for performing a respective medical intervention, as described herein.
For the above cases, the specific ML models may include a combination of specific ML models and rule based models.
For the above cases, the combined prioritization component may be implemented as an ML model and/or aggregation code.

Optionally, validation dataset(s) and/or testing dataset(s) may be generated for testing and/or validating the ML models and/or combined prioritization component.

At 322, one or more features described with reference to 302-322 are iterated, for example, for updating the training datasets for generated updated ML models in response to new and/or changes to the EMR of the subjects.

Iterations may be automatic and/or manual.

Iterations for updating of models and/or model types may be triggered, for example, by one or more of: date, new data, performance degradation of the ML model themselves, change in infrastructure demands (e.g., expected output types, etc.), and/or user requests (e.g., change explanations).

Referring now back to FIG. 4, at 402, a risk parameter for each subject is accessed. The risk parameter is indicative of risk of a certain clinical outcome (e.g., heart attack, stroke, hospitalization) and/or a diagnosis of a certain clinical diagnosis (e.g., cancer, type 2 diabetes).

There may be multiple risk parameters for each subject, each indicative of risk of a certain clinical outcome and or a diagnosis of a clinical outcome. In such a case, a respective maximal recommended time interval for reducing or preventing the target clinical outcome is computed for each subject for each risk parameter.

The risk parameter may be computed, for example, as one or more of:
An outcome of a risk ML model (e.g., as described herein) fed an input of data extracted from EMRs of subjects, and trained on a training dataset of sets of data extracted from sample EMRs of sample subjects, each set of data labeled with a respective ground truth label of the risk parameter.
A score of the subject on a certain specific priority list and/or on the combined priority lists (computed as described herein). For example, subjects on a list of risk of stroke are assigned risk scores indicating ranking and/or likelihood of developing stroke.
Manually entered, for example, by a user, such as a clinician that took a history and performed a physical examination of a patient and entered a “high risk” category for the patient
A set of rules applied to data, such as EMR data. For example, subjects with a combination of blood test values above a certain threshold and/or within certain ranges re classified into a certain value of the risk parameter.

At 404, mapping code is trained and/or provided for mapping the respective risk parameter to a certain maximal recommended time interval for performing a corresponding medical intervention on the respective subject for reducing or preventing a target clinical outcome.

The mapping code may be implemented as a mapping ML model. The mapping ML model may be trained on a training dataset of sample sets of subject parameters extracted from the EMR of each sample subject, and optional risk parameter(s) for each sample subject, where each sample set is labeled with a ground truth indication of a respective maximal recommended time interval (e.g., manually provided and/or computed by code).

The risk parameter may be represented by internal weights of the mapping ML model, for example, weights of neurons of an intermediate layer of a neural network ML model implementation. In such implementation, the risk parameter may not necessarily be explicitly extracted and provided. Alternatively or additionally, the risk parameters of multiple subjects are inputted (e.g., as a vector, multi-dimensional dataset) into the mapping ML model. Alternatively or additionally, the subject parameters and risk parameters are inputted into the mapping ML model.

Alternatively or additionally, the mapping code is implemented as a set of rules that are dynamically adjustable by a user via a GUI presented on a display and/or prefixed.

Alternatively or additionally, the mapping code is implemented as predefined mapping function, for example, a fixed set of rules.

At 406, for respective subjects, the respective risk parameter is mapped by the mapping code to the certain maximal recommended time interval for performing the corresponding medical intervention on the respective subject for reducing or preventing the target clinical outcome.

The target clinical outcomes may correspond to the target clinical outcomes of the specific priority lists, the combined priority lists, and/or be unrelated to the priority lists.

The maximal recommended time interval may be, for example, a week, a month, two months, three months, six months, or a year. The medical intervention is to be performed within the time interval, but no later than the end of the time interval.

The maximal recommended time interval may be a frequency for performing the medical intervention at temporal intervals that do not exceed the certain maximal recommended time interval.

The medical intervention may be, for example, an evaluation of the medical record of the subject by a health practitioner to determine if changes to the health management of the subject is required, for example, quarter yearly evaluations. The evaluation may be performed without presence of the subject. In another example, the medical intervention may be, for example, blood tests for monitoring a medical condition (e.g., HbA1c for type 2 diabetes), cancer screening (e.g., mammography every 2 years), and the like.

The risk parameter may be represented, for example, as/by a classification category (e.g., high, medium, low), a numerical probability value, and/or a risk score in a defined range (e.g., 0-100). For the case of the defined range, different sub-ranges are mapped to different maximal recommended time intervals. For example, for risk value indicating likelihood of hospitalization in the following year, for a risk percentile 75-100: follow up at least every 6 months; for a risk percentile 60-75: follow up at least annually; for a risk percentile 40-60: follow up at least every two years; and for a risk percentile 0-40: follow up at least every three years.

The mapping is performed by the mapping code. For example, by applying the set of rules. In another example, by feeding the risk parameters and/or feeding subject parameters extracted from an EMR of the respective subject into the trained mapping ML model that generates an outcome of the certain maximal recommended time interval.

At 408, candidate appointments for performing the corresponding medical intervention that have dates falling within and not exceeding the certain maximal recommended time interval may be presented within a GUI presented on a display. The GUI may block scheduling the next intervention time to later than the maximal recommended time interval. For example, when the maximal recommended time interval is 6 months, 1 month, 3 months, and 6 months are provided as options, while later dates (e.g., 9 months, 12 months) are blocked.

The appointment, for a respective subject, for performing the corresponding medical intervention at a date falling within and not exceeding the certain maximal recommended time interval, may be set via the GUI, and/or automatically.

Optionally, multiple future timeslots are reserved on an electronic calendar of the healthcare provider that performs the medical intervention. Each respective timeslot may be reserved for reviewing the EMR of the respective subject at frequency of dates each falling within and not exceeding the certain maximal recommended time interval from a previously scheduled timeslot.

At 410, the corresponding medical intervention is performed before the maximal recommended time interval, optionally at the prescheduled appointment. For example, a review of the EMR of the respective subject by a healthcare provider without the presence of the subject. In another example, a laboratory test and/or medical imaging examinant of the subject.

At 412, a determination may be made that the respective subject did not attend the appointment, and/or the healthcare provider did not perform the medical intervention at the selected appointment. Another appointment for performing the corresponding medical intervention at a date falling within and not exceeding the certain maximal recommended time interval may be scheduled, optionally automatically.

At 414, one or more features described with reference to 402-412 may be iterated, for example, in response to new data of the subject which may impact the value of the risk parameter.

Iterations may be automatic and/or manual.

Iterations may be triggered, for example, by one or more of: date, new data, performance degradation, infrastructure changes, and/or user requests.

In an example, after a healthcare provider performs the review of an EMR of the respective subject without the presence of the subject, within the maximal recommended time interval, the following may be iterated: an update of the risk parameter(s) for the respective subject is computed. An updated mapping of the update of the risk parameter(s) to an updated maximal recommended time interval for performing another review of the EMR of the respective subject without the presence of the subject. Future timeslot(s) for performing another review of an updated EMR of the respective subject is presented within the GUI. The future timeslot(s) fall within and do not exceed, the certain maximal recommended time interval from a date of the previously performed review of the EMR of the respective subject. Future timeslot(s) are scheduled in response to a selection of one of the future timeslots.

Referring now back to FIG. 5, GUI 502 presents examples of specific priority lists, in accordance with some embodiments of the present invention. A list of specific priority lists 504 are presented under the heading “Priority lists”. Examples of priority lists are for the following target clinical outcomes: “Missed Critical Referrals”, “Deterioration from chronic illness”, “COVID vaccinations”, “Diabetes”, “Hypertension”, and “Cardiovascular disease”. The number of patients on each respective priority list may be provided.

The user may select a certain specific priority list to view details of the patients on that list. For example, as shown, the “Missed Critical Referrals” specific priority list is selected.

A severity score 506 is assigned to the patient for each specific priority list, as described herein. The severity score may be used for ranking the patient within each specific priority list. Each severity score may represent a risk parameter that may be mapped to a risk category, for example, “Very high risk group”, and/or for which a target date for review of the patient's clinical file 508 (i.e., the maximal time interval for follow up) may be automatically set, as described herein.

Optionally, a personalized recommendation for treatment and/or evaluation by a specific medical intervention 510 is presented. For example, as shown, Patient A requires a breast biopsy, Patient B requires a Cardiac CT, Patient C requires Cardiac catheterization, etc. . . . . Optionally, referral dates 512 are provided for each patient for each recommended specific medical intervention.

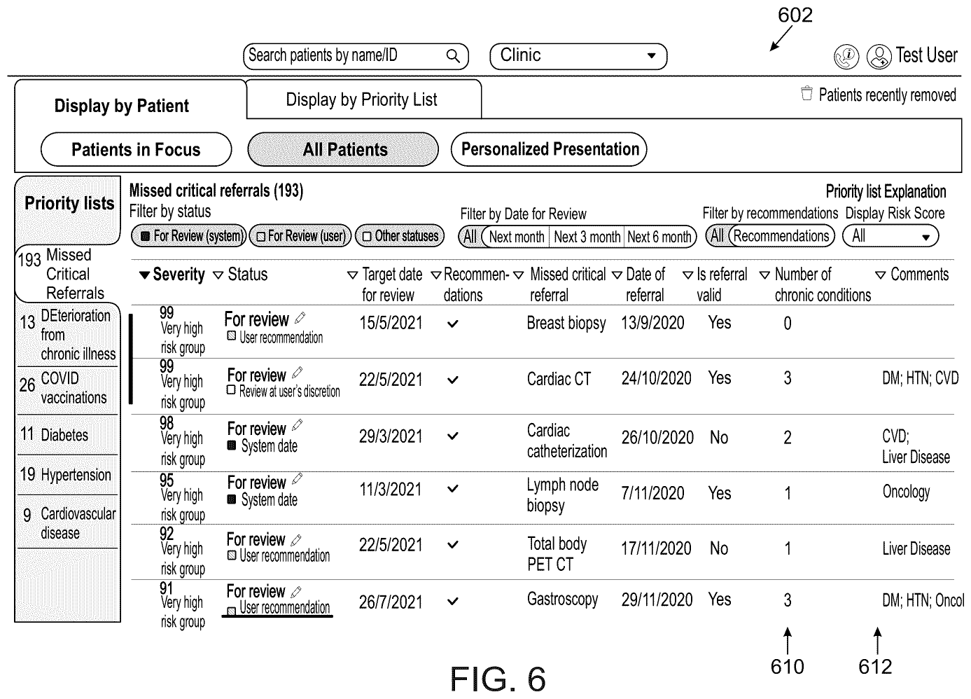

Referring now back to FIG. 6, GUI 602 corresponds to GUI 502 described with reference to FIG. 5, with a further horizontal scroll to the right, displaying additional data for patients on the selected specific priority list, in accordance with some embodiments of the present invention. GUI 602 may further display a number of chronic conditions 610 of each patient, and a list 612 of what the chronic conditions are.

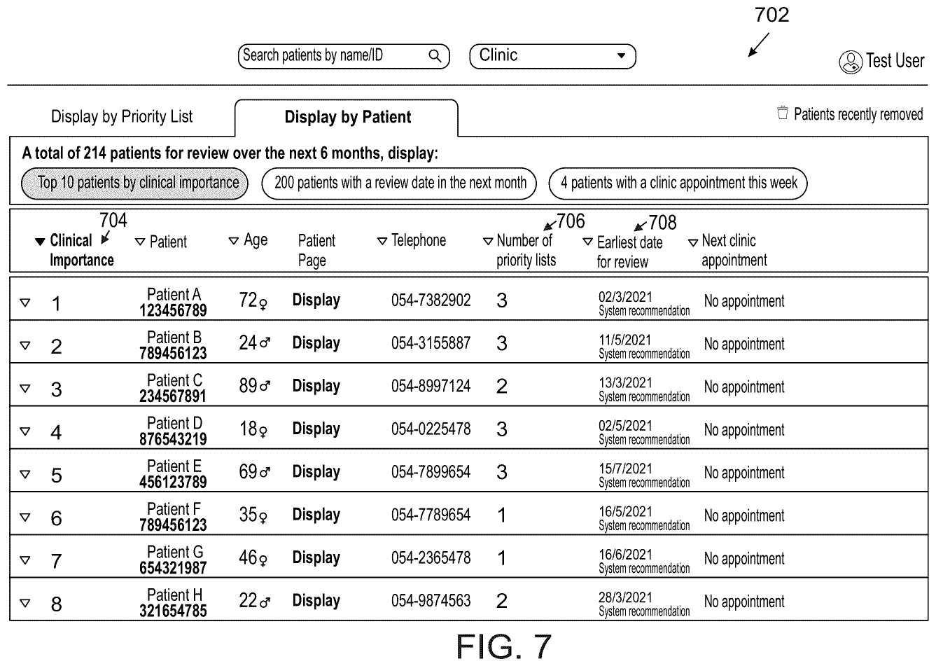

Referring now back to FIG. 7, GUI 702 depicts an exemplary combined priority list, in accordance with some embodiments of the present invention. Patients are ranked by clinical importance 704, for example, by a combined risk score as described herein. It is noted that the relative ranking of the patients is shown, while the absolute value of the combined risk score is not necessarily depicted. GUI 702 may further present a number of specific priority lists 706 each patient is listed on. The “Earliest data for review” column 708 indicates the earliest target date for review (out of the target dates for all specific priority lists in which the patient is included).

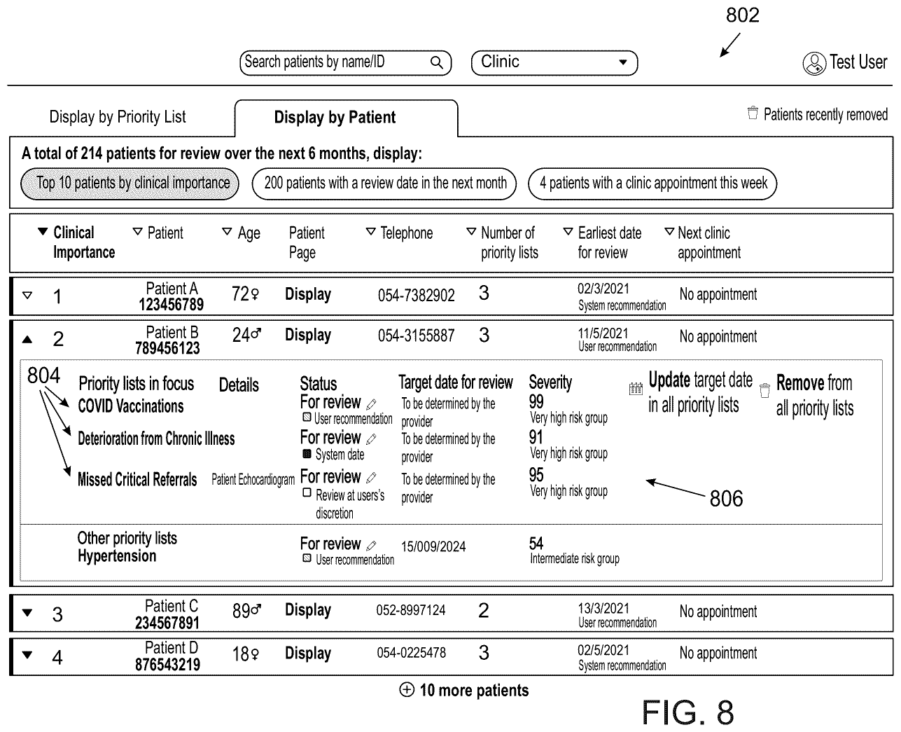

Referring now back to FIG. 8, GUI 802 depicts GUI 702 of FIG. 7 expanded for “Patient B” (e.g., by clicking on the row corresponding to Patient B), presenting multiple specific priority lists 804 that include Patient B, which are combined and/or aggregated to determine the ranking of Patient B on the combined priority list, in accordance with some embodiments of the present invention. For example, as shown, Patient B is on the following priority lists for the target clinical outcomes of “COVID vaccinations”, “Deterioration from Chronic Illness”, and “Missed Critical referrals”. A severity score 806 is assigned to the patient for each specific priority list, as described herein. The severity score may be used for ranking the patient within each specific priority list. A combination of the severity scores may be used for ranking the patient within the combined priority list, as described herein. Each severity score may represent a risk parameter that may be mapped to a risk category, for example, “Very high risk group”, and/or for which a maximal time interval for follow up may be determined, as described herein.

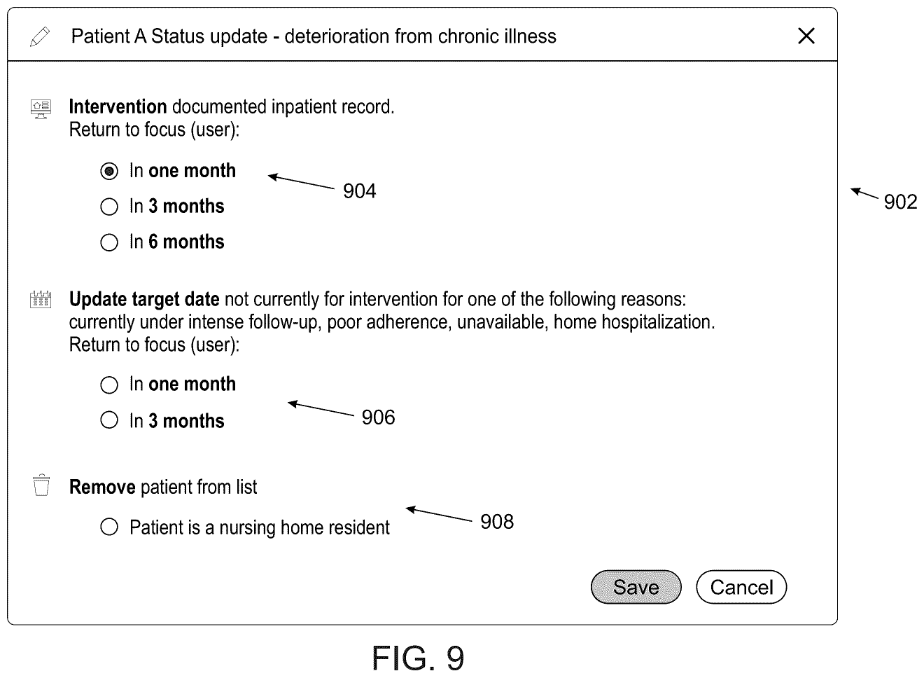

Reference is now made to FIG. 9, which is a GUI 902 presenting a maximal recommended time interval 904 for performing a review of the clinical file of “Patient A” for reducing or preventing the target clinical outcome of “Deterioration from chronic illness”, in accordance with some embodiments of the present invention. The maximal recommended time interval 904 is computed to be 6 months based on risk parameters of Patient A, as described herein. Options for reviewing the clinical file may be presented, for example, one month, three months, and six months.

GUI 902 may present other options, for example, an option 906 to ask to be reminded later within the maximal recommended time interval, for example, one month, and 3 months. Another option presented by GUI 902 is to remove the patient from the list 908 due to a reason which may be selected, as shown.

Patient A may be selected from a specific priority list and/or from a combined priority list, as described herein.

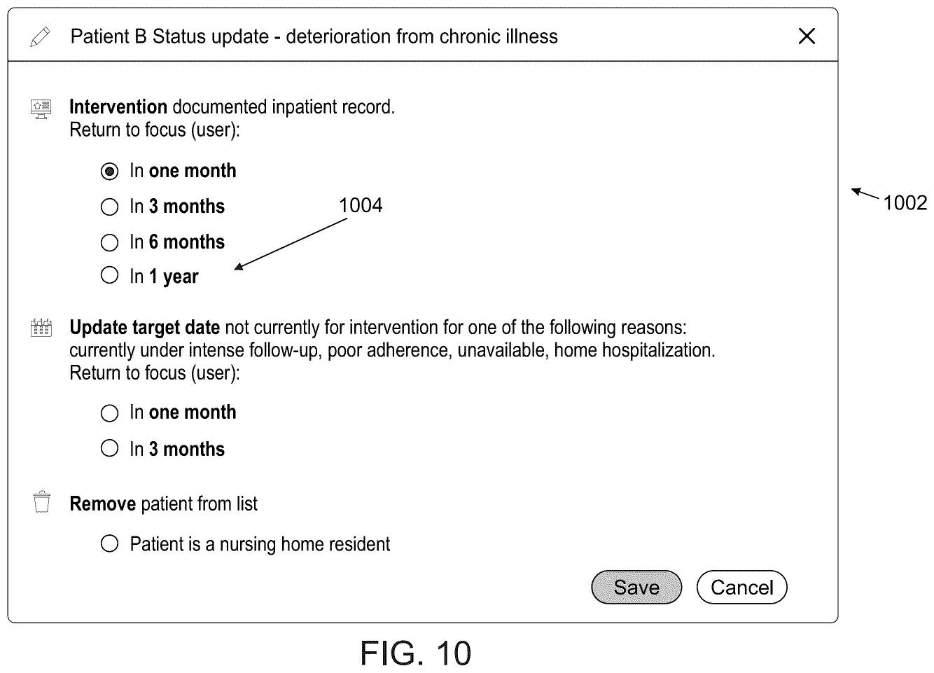

Reference is now made to FIG. 10, which is a GUI 1002 presenting a maximal recommended time interval 1004 for performing a review of the clinical file of “Patient B” for reducing or preventing the target clinical outcome of “Deterioration from chronic illness”, in accordance with some embodiments of the present invention. GUI 1002 is similar to GUI 902 of FIG. 9, however attention is drawn to the additional “1 year” option 1004 for the maximal recommended time interval, which does not appear in GUI 902 for Patient A. This is because Patient B is considered lower risk that patient A (computed using respective risk parameters and corresponding mappings, as described herein), which provides the option for a longer time between follow-up review sessions of the clinical file.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant ML models will be developed and the scope of the term ML model is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A system for training a plurality of machine learning models and providing a combined prioritization component for dynamic prioritization of target subjects for at least one of evaluation, and treatment, comprising:
at least one processor executing a code for:
accessing electronic medical records (EMR) of a set of a plurality of subjects;
for at least one of each of a plurality of specific medical interventions that are resource limited, and for each respective target clinical outcome:
accessing a respective specific priority list of a respective sub-set of the plurality of subjects scheduled in a prioritized sequence for at least one of treatment and evaluation for at least one of the respective target clinical outcome and by the respective specific medical intervention;
creating a respective specific training dataset that includes data extracted from the EMRs of at least the respective sub-set of the plurality of subjects labelled with the specific priority list; and
training a respective specific machine learning model on the respective specific training dataset for generating an outcome of a respective target specific priority list of a sub-set of target subjects for prioritized at least one of evaluation and treatment for the at least one of respective target clinical outcome and by the respective specific medical intervention, in response to an input of data extracted from EMR of at least the sub-set of target subjects;
accessing an intermediate component for computing a plurality of weighted specific priority lists by assigning a respective preselected global list weight to each respective target specific priority list outcome of respective specific machine learning models,
wherein each respective preselected global list weight indicates a magnitude of prioritization of the respective target specific priority list;
wherein the same respective preselected global list weight is applied to all scores of all subjects of the same respective target specificity priority list,
wherein each respective global weight of each respective priority list is adjustable;
accessing a combined prioritization component for generating an outcome of a target combined priority list of target subjects for prioritized at least one of evaluation and treatment in response to an input of the plurality of weighted specific priority lists; and
providing the plurality of specific machine learning models, the intermediate component, and the combined prioritization component,
wherein the plurality of specific machine learning models and the combined prioritization component generate a respective numerical score for each respective subject, and the specific priority lists and the combined priority list are created by ranking subjects according to respective numerical scores,
wherein the combined prioritization component computes, for each respective subject of the plurality of target subjects, a respective weighted score by multiplying each respective numerical score assigned to the respective subject on each respective priority list by the respective global list weight assigned to the respective list.

2. The system of claim 1, wherein respective preselected global list weights indicating the magnitude of prioritization of the respective target specific priority lists comprise a numerical value.

3. The system of claim 1, wherein respective preselected global list weights indicating the magnitude of prioritization of the respective target specific priority list are selected according to a geographical location.

4. The system of claim 1, wherein respective preselected global list weights indicating the magnitude of prioritization of the respective target specific priority list are defined according to a set of rules.

5. The system of claim 1, wherein respective preselected global list weights indicating the magnitude of prioritization of the respective target specific priority list are fixed within a defined range of values.

6. The system of claim 1, wherein respective preselected global list weights indicating the magnitude of prioritization of the respective target specific priority list are adjustable to different values by different users.

7. The system of claim 1, wherein the combined prioritization component comprises a machine learning model, and further comprising code for:
creating a main training dataset that includes a plurality of specific priority lists obtained as outcomes of a plurality of specific machine learning models, labelled with a respective combined priority list of the set of the plurality of subjects for at least one of treatment and evaluation; and
training the combined prioritization component on the main training dataset.

8. The system of claim 1, wherein the combined prioritization component comprises aggregation code that when executed by a processor aggregates, for each subject of the plurality of subjects, the respective numerical scores of the plurality of specific priority lists into an aggregated score; and generates the target combined priority list by ranking the plurality of subjects according to respective aggregated scores.

9. The system of claim 8, wherein each of the plurality of priority lists is assigned a respective weight, and wherein aggregating comprises, computing, for each respective subject a respective weighted score by multiplying each respective numerical score assigned to the respective subject on each respective priority list by the respective weight assigned to the respective list, and ranking the plurality of subjects according to respective weighted scores, the combined priority list comprising the ranked weighted scores.

10. The system of claim 1, wherein at least one of a plurality of the target specific priority lists and the target combined priority list are time correlated priority lists, denoting, for each respective rank within each respective time correlated priority list, a maximal recommended time interval for performing a corresponding medical intervention on the respective subject at the respective rank for reducing or preventing a target clinical outcome.

11. (canceled)

12. The system of claim 1, wherein at least one of a plurality of the target specific priority lists and the target combined priority list are time correlated priority lists, wherein each respective rank within each respective time correlated priority list denotes a recommended maximal time interval for performing a corresponding medical intervention on the respective subject for an effective allocation of resources for performing the medical intervention based on an input of a schedule of availability of resources for performing each respective corresponding medical intervention for each of a plurality of time intervals.

13. The system of claim 12, wherein at least one of each respective specific training dataset and a main training dataset further includes a schedule of availability of resources for performing each respective corresponding medical intervention for each of a plurality of time intervals, and the respective machine learning model is trained for generating a respective time correlated priority list in response to an input of a schedule of time correlated availability of the respective resource for performing the respective medical intervention.

14. The system of claim 1, wherein the respective specific training dataset further includes, for each respective sub-set of the plurality of subjects, an indication of risk of at least one of a respective clinical outcome and/or a diagnosis of a respective clinical diagnosis,
wherein each respective specific machine learning model is for at least one of a respective clinical outcome and for the respective clinical diagnosis
wherein each respective specific machine learning model is trained to generate the outcome of the respective target priority list of the sub-set of target subjects at risk for the at least one of clinical outcome and diagnosed with the respective clinical diagnosis, for prioritized at least one of evaluation and treatment for the at least one of respective clinical outcome, respective clinical diagnosis, and target clinical outcome by the respective specific medical intervention.

15. The system of claim 1, further comprising code for:
receiving a set of target EMRs of a plurality of target subjects;
feeding the set of target EMRs into the plurality of specific machine learning models;
obtaining a plurality of specific priority lists as outcomes of the plurality of specific machine learning models;
feeding the plurality of specific priority lists into the combined prioritization component; and
obtaining a combined priority list as an outcome of the combined prioritization component.

16. The system of claim 15, further comprising code for:
sequentially at least one of treating and evaluating the plurality of target subjects for a plurality of respective target clinical outcomes by the plurality of specific medical interventions according to a prioritized order defined by the plurality of specific priority lists; and
sequentially at least one of treating and evaluating the plurality of target subjects according to the prioritized order defined by the combined priority list.

17-19. (canceled)

20. The system of claim 1, further comprising code for generating instructions for at least one of treating and evaluation target subjects by a main medical intervention according to the target combined priority list.

21-23. (canceled)

24. The system of claim 1, further comprising code for outputting for each respective subject, at least one of: the respective numerical score computed by the combined prioritization component, and a ranking of the respective subject on the combined priority list.

25. The system of claim 1, wherein each respective weight of each respective priority list is dynamically adjustable by a user via a user interface.

26. A system for prioritization of target subjects for at least one of treatment and evaluation, comprising
at least one processor executing a code for:
accessing EMRs of a set of a plurality of subjects;
feeding the EMRs into each of a plurality of specific machine learning models;
obtaining a plurality of specific priority lists as outcomes of the plurality of specific machine learning models, wherein each respective specific priority list includes a respective sub-set of the plurality of subjects scheduled in a prioritized sequence for at least one of treatment and evaluation for at least one of a respective target clinical outcome and by a respective specific medical intervention;
feeding the plurality of specific priority lists into an intermediate component that assigns a respective preselected global list weight to each respective target specific priority list,
wherein each respective preselected global list weight indicates a magnitude of prioritization of the respective target specific priority list,
wherein the same respective preselected global list weight is applied to all scores of all subjects of the same respective target specificity priority list,
wherein each respective preselected weight of each respective priority list is adjustable;
obtaining a plurality of weighted specific priority lists from the intermediate component;
feeding the plurality of weighted specific priority lists into combined prioritization component; and
obtaining a combined priority list as an outcome of the combined prioritization component, wherein the combined priority list is of the plurality of subjects indicating priority for at least one of evaluation and treatment,
wherein the plurality of specific machine learning models and the combined prioritization component generate a respective numerical score for each respective subject, and the specific priority lists and the combined priority list are created by ranking subjects according to respective numerical scores,
wherein the combined prioritization component computes, for each respective subject of the plurality of target subjects, a respective weighted score by multiplying each respective numerical score assigned to the respective subject on each respective priority list by the respective global list weight assigned to the respective list.

27-44. (canceled)

45. A system for dynamic prioritization of target subjects for at least one of evaluation and treatment, comprising:
at least one processor executing a code for:
accessing a plurality of specific priority lists of a respective sub-set of a plurality of subjects scheduled in a prioritized sequence for at least one of treatment and evaluation for at least one of a respective target clinical outcome and by a respective specific medical intervention,
wherein each subject in each of the plurality of specific priority lists is associated with a respective numerical score indicative of risk of a respective clinical outcome;
wherein each of the plurality of priority lists is assigned a respective global list weight that is adjustable;
wherein each respective preselected global list weight indicates a magnitude of prioritization of the respective target specific priority list;
wherein the same respective preselected global list weight is applied to all scores of all subjects of the same respective target specificity priority list,
computing, for each respective subject of the plurality of subjects, a respective weighted score by multiplying each respective numerical score assigned to the respective subject on each respective priority list by the respective global list weight assigned to the respective list; and
generating a combined priority list by ranking the plurality of subjects according to respective weighted scores, wherein the combined priority list comprising the ranked weighted scores.
